(19) United States
(12) Patent Application Publication
(10) Pub. No.: US 2023/0293872 A1
(43) Pub. Date: Sep. 21, 2023

(54) METHOD FOR FABRICATING A CRYOMICRONEEDLE AND A CRYOMICRONEEDLE FABRICATED ACCORDING THERETO

(71) Applicant: City University of Hong Kong, Kowloon (HK)

(72) Inventors: Chenjie Xu, Kowloon (HK); Mengjia Zheng, Kowloon (HK)

(21) Appl. No.: 18/093,990

(22) Filed: Jan. 6, 2023

Related U.S. Application Data
(63) Continuation-in-part of application No. 17/443,523, filed on Jul. 27, 2021.
(60) Provisional application No. 63/296,915, filed on Jan. 6, 2022.

(51) Int. Cl. A61M 37/00; C12N 5/00; A61K 35/12; A61K 39/00

(52) U.S. Cl. CPC A61M 37/0015; C12N 5/0062; A61K 35/12; A61K 39/4643; A61M 2037/0053

(57) ABSTRACT

A method for fabricating a cryomicroneedle, includes the steps of: providing a microneedle scaffold including a plurality of pores; providing a suspension including a biological agent; loading the biological agent into the microneedle scaffold by immersing the microneedle scaffold in the suspension to form a loaded microneedle scaffold; and freezing the loaded microneedle scaffold to provide the cryomicroneedle. A cryomicroneedle prepared according to the method above and methods for using such a cryomicroneedle are described as well.

### CROSS REFERENCE TO RELATED APPLICATIONS

This application claims the benefit of U.S. Provisional Patent Application No. 63/296,915, filed Jan. 6, 2022. This application is also a Continuation-in-Part of U.S. patent application Ser. No. 17/443,523, filed Jul. 27, 2021.

### TECHNICAL FIELD

The present invention generally relates to the technical field of microneedles. More specifically, the present invention relates to a method for fabricating a cryomicroneedle and uses for a cryomicroneedle.

### BACKGROUND

The intradermal delivery of therapeutic cells has great potentials for treating both local and systematic diseases (e.g. mesenchymal stem cells (MSCs) for wound healing, dendritic cells for immunotherapy, beta cells for diabetes). The key here is the precise delivery of cells at the desired depth and location for the optimal response and outcome. Microneedles (MNs) are minimized hypodermic needle arrays and considered as a powerful platform for the transdermal delivery of the therapeutic cells.

A few MN platforms have been developed for delivering cells. For example, Chua et al., replaced the conventional catheter needle with a micro-scale hollow metal needle to reduce the risk of microstrokes and further inflammatory reactions.^{[1]} Gualeni et. al., adopted a hollow MN to transplant noncultured epidermal cell suspension for vitiligo treatment.^{[2]} However, these hollow MNs for cell infusion require the assistance of extra devices such as an insertion device to assist in precise delivery, and a syringe or reservoir to store the cell suspension.

There are also a few new MN platforms for cell delivery without the assistance of the extra device. For example, Lee et. al., developed a hydrogel MN with an outer poly(lactic-co-glycolic) (PLGA) protective shell to deliver MSCs for wound regeneration.^{[3]} Chen et. al., seeded human keratinocytes and human follicle dermal papilla cells on the surface of solid poly-methyl methacrylate (PMMA) or metal MN patches.^{[4]} Subsequently, the coated cells could be transplanted into the hydrogel or the targeted tissues within 3 days. Li et. al., seeded chimeric antigen receptor (CAR) expressing T cells on the surface of porous MNs and implanted the CAR T cells for direct intratumoral injection. However, these cell-loaded MN platforms still require fresh preparation and immediate application of the cell products to preserve cell viability and functionality.

Other attempts to fabricate MNs and/or to transdermally deliver cells include U.S. Pat. No. 9,040,087 B2, entitled “Frozen Compositions and Methods for Piercing a Substrate”, to Boyden, et al., published 26 May 2015, proposes MNs as the frozen piercing implements. However, this patent does not teach the use of the claimed MNs for cell delivery purposes.

US Patent Application No. 2010/0187728 A1, entitled “Systems, Devices, and Methods for Making or Administering Frozen Particles”, to Boyden, et al., published on 29 Jun. 2010, discloses delivery of frozen particles. However, it does not mention a cryogenic formulation for the purpose of enhancing cell viability. Also, it does not disclose a microneedle platform for transdermal cell delivery in the application.

PCT application WO 2018/017674 A1, entitled “Methods, Compositions, and Devices for Drug/Live Cell Microarrays”, to Pathak, published on 25 Jan. 2018, discloses live cell delivery based on “array in array” of hollow microneedles. However, the feasibility of its solid-state delivery of cells is not verified in the application. It discloses a polymer-based formulation, yet the formulation does not appear to enhance cell viability. U.S. Pat. No. 10,624,865 B2, entitled “Methods, Compositions, and Devices for Drug/Live cell microarrays”, to Pathak, published on 21 Apr. 2020, is a continuation-in-part of WO 2018/017674 A1, entitled “Methods, Compositions, and Devices for Drug/Live cell microarrays”, to Pathak, published on 25 Jan. 2018, and has the same technical problems.

PCT application WO 2015/132568 A1, entitled “Microneedle Based Cell Delivery”, to Birchall, et al., published on 11 Sep. 2015, discloses transdermal cell delivery based on hollow microneedle device, where cell delivery through a microneedle is achieved by using a liquid cell suspension.

PCT application WO 2010/040271 A1, entitled “Phase-Transition Polymeric Microneedles”, to Jin, published on 15 Apr. 2010, discloses a freeze-thaw treatment to crosslink a microneedle matrix. However, it does not mention the use of the crosslinked microneedle matrix for cell delivery purposes.

Chinese patent application No. CN 112516452 A, entitled “Frozen Microneedle Array, Preparation Method Therefor and Application of Frozen Microneedle Array”, to Zhao, et al., published on 19 Mar. 2021, discloses a method of preparing a frozen microneedle array. However, it does not mention any porous scaffold for the dipping and loading of a cell suspension. Also, it does not mention the use of the frozen microneedle array for cell delivery purposes.

The previous generation of the inventors' cryomicroneedle (cryoMN) platform technology allows the package of cells into MNs in advance and the direct usage in situ. (U.S. patent application Ser. No. 17/443,507) This device can be stored for at least 6 months and transported in a container with dry-ice or liquid nitrogen, and has been tesonted for transdermal delivery of ovalbumin-pulsed dendritic cells (OVA-DCs) for DC-based vaccination and immunotherapy. Vaccination using OVA-DCs loaded cryoMNs effectively induced antigen-specific immune responses and suppressed tumor growth in mice, comparable to those with conventional injection of OVA-DCs. However, such cryoMNs are fabricated through a stepwise cryogenic molding of a cell suspension before they are demolded and stored in the liquid nitrogen/−80° C. freezer. This multi-step process is laborious and time-consuming. It also increases the potentials of contaminations and cell damages during the handling.

As such, there remains a need for a MN platform which may be fabricated via a simplified process while solving the above-noted technical problems with existing MN platforms, including, but not limited to, enhanced cell viability, enhanced cell activity, enhanced cell functionality, diversity in delivered cells, easy use, easy preparation, easy storage, easy transportation, etc. There also exists a need for in situ preparation of cell loaded MNs without aid of extra equipment that facilitates the translation for onsite transplantation of autologous cells in clinics.

### SUMMARY OF THE INVENTION

An aspect of the present invention relates to a method for fabricating a cryomicroneedle, including: providing a microneedle scaffold including a plurality of pores; providing a suspension including a biological agent; loading the biological agent into the microneedle scaffold by immersing the microneedle scaffold in the suspension to form a loaded microneedle scaffold; and freezing the loaded microneedle scaffold to provide the cryomicroneedle.

Another aspect of the present invention relates to a cryomicroneedle prepared according to the method of the present invention.

Without intending to be bound by theory, it is believed that the present invention provides a simplified process for fabricating a cryomicroneedle with enhanced cell viability, enhanced cell activity, enhanced cell functionality, diversity in delivered cells, easy use, easy preparation and easy transportation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

It will be convenient to further describe the present invention with respect to the accompanying drawings that illustrate possible arrangements of the invention. Other arrangements of the invention are possible and consequently, the particularity of the accompanying drawing is not limiting and is not to be understood as superseding the generality of the preceding description of the invention.

FIG. 1 shows a schematic of the fabrication and application of an embodiment of a cryomicroneedle according to an embodiment of the present invention;

FIG. 2A shows a schematic of a fabrication process of a microneedle scaffold according to an embodiment of the present invention;

FIG. 2B shows a photo of an embodiment of the MeHA MN scaffold (Scale bar: 2 mm);

FIG. 2C shows a SEM image of an embodiment of the MeHA MN scaffold (Scale bar: 250 μm);

FIG. 3A illustrates optimization studies of hydrogel concentration (i.e. 2, 4, 5, 6 wt. %) for the MN fabrication using MeHA with the molecule weight of 300 kDa (300-MeHA) in an embodiment of the invention herein;

FIG. 3B illustrates optimization studies of hydrogel concentration (i.e. 2, 4, 6, 8 wt. %) for the MN fabrication using MeHA with the molecule weight of 48 kDa (48-MeHA) in an embodiment of the invention herein;

FIG. 3C illustrates optimization studies of the cross-linking time for the porous MN scaffolds made of 4 wt. % of 300-MeHA in an embodiment of the invention herein;

FIG. 3D illustrates optimization studies of the cross-linking time for the porous MN scaffolds made of 6 wt. % of 48-MeHA;

FIGS. 4A-E illustrate the optimization of embodiments of the cryoprotective medium herein featured with hydrogel-forming polymers and DMSO: the viability of human skin fibroblasts after cryopreservation in cryogenic solutions containing various ratio of DMSO (i.e. 0, 1, 2%, v/v) and different concentrations (i.e. 0, 0.25, 0.50, 1.0, 2.5 wt. %) of a hydrogel polymer, and FIG. 4A shows results of polyethylene glycol (PEG), where *P≤0.05, **P≤0.01, ***P≤0.001, ns: P>0.05, no significant difference (ns);

FIG. 4B shows results of hydroxyethyl starch (HES);

FIG. 4C shows results of methyl cellulose (MC);

FIG. 4D shows results of carboxymethyl cellulose (CMC);

FIG. 4E shows the viability of fibroblasts, HaCaT, A375, and hMSCs after cryopreservation in cryogenic solutions containing hydrogel-forming polymers;

FIGS. 5A-F illustrate studies of cell-loaded cryoMNs according to an embodiment of the present invention for the loading capacity and cell viability, and, specifically, FIG. 5A shows cryoMNs before and after thawing (scale bar: 2 mm);

FIG. 5B shows 3D reconstruction;

FIG. 5C shows a top view of confocal image showing the cell release from cryoMNs into the agarose gel phantom (Scale bar: 100 μm);

FIGS. 5DA through 5DD show the z projection of confocal image;

FIG. 5E shows quantification of the cells released from a single cryoMN patch loaded with different cell densities ranging from 2.5×10\hat{ }6, 5.0×10\hat{ }6, 7.5×10\hat{ }6 to 1.0×10\hat{ }7 cells/mL;

FIG. 5F shows the live/dead staining of cells released from cryoMNs (scale bar: 200 μm);

FIG. 6A shows in vitro examination of hMSCs delivered using cryoMNs according to an embodiment of the present invention for wound healing. *P≤0.05, **P≤0.01, ***P≤0.001, ****P≤0.0001, and, specifically, FIG. 6A shows the experiment setup for the scratch assay (wound healing model);

FIG. 6B shows quantification of the wound closure for each treatment group;

FIG. 6C schematically shows how hMSCs-loaded MNs are used in promoting HUVEC (Human umbilical vein endothelial cell) vascularization in the endothelial cell tube formation assay;

FIG. 6D shows the quantification of the tube length;

FIG. 6E shows the quantification of the number of branching nodes;

FIG. 6F shows the quantification of the number of tubes for each treatment;

FIGS. 7A-E show in vivo vaccination using ovalbumin-pulsed dendritic cells (OVA-DCs) loaded cryoMNs. *P≤0.05, **P≤0.01, ***P≤0.001, ns: P>0.05, no significant difference (ns), and, specifically, FIG. 7A schematically shows the timeline of vaccination with OVA-DCs loaded cryoMNs, which is compared with subcutaneous (s.c.) injection;

FIG. 7B shows the photos before and after administration of 2 patches of OVA-DCs on the shaved back of mice;

FIG. 7C shows the recovery of mouse skin after removal of cryoMNs, in which skin is gradually recovered within 60 minutes (scale bar: 1 cm);

FIG. 7D shows the histological analysis of penetration depth using cryoMNs (scale bar: 100 μm);

FIG. 7E shows representative plots of CD11c+CD86+ and CD11c+ MHCII+ DCs insides the draining lymph nodes using cryoMNs and s.c. injection;

FIG. 7F and FIG. 7G show the quantitative analysis of CD11c+CD86+ and CD11c+ MHCII+ DCs insides the draining lymph nodes, respectively;

FIG. 7H shows proliferation of splenocytes from vaccinated mice after restimulation using OVA. Each group has three independent animals;

FIG. 8A shows the ¹H NMR spectrum of MeHA with a molecular weight of 48 kDa (48-MeHA);

FIG. 8B shows the ¹H NMR spectrum of MeHA with a molecular weight of 300 kDa (300-MeHA);

FIG. 9 shows SEM image of the original stainless-steel template at 2 different magnifications;

FIG. 10 shows that the cryogenic mediums formulated with alginate, gelatin, chitosan, hyaluronic acid (HA) with 1% DMSO and 2% DMSO (v/v) do not significantly improve the cell viability of dermal fibroblasts during cryopreservation (Scale bar: 250 μm);

FIGS. 11A-D show the safety of cryogenic medium formulated with PEG, HES, CMC and MC, and, specifically

FIG. 11A shows the safety of the formulation towards dermal fibroblasts;

FIG. 11B shows the safety of the formulation towards HaCaT (human keratinocytes);

FIG. 11C shows the safety of the formulation towards A375 (human melanoma cells);

FIG. 11D shows the safety of the formulation towards hMSCs, wherein each type of cell is incubated with the culture medium supplemented with polymers and DMSO at 37° C. for 24 hrs;

FIG. 12 shows the thawing behavior of cryoMNs loaded with CMC cryogenic medium according to an embodiment of the present invention after being exposed to room temperature (24° C.) within 2 min;

FIG. 13 shows the penetration behavior of cryoMNs with four types of polymer cryogenic medium (i.e., PEG, HES, MC and CMC) tested on ex vivo porcine ear skin (Scale bar: 250 μm);

FIG. 14 shows the live (green)/dead (red) staining images of the hMSCs and fibroblasts after cryopreservation and release from the cryoMNs (Scale bar: 100 μm);

FIG. 15 shows the microscopic images of the wound area in the scratch assay (wound healing model, FIG. 6A) for 24 hrs and 48 hrs after treated with low serum medium (1% FBS), 20 ng/mL TGF-β1, blank cryoMNs, and hMSCs-loaded cryoMNs (Scale bar: 250 μm);

FIG. 16 shows the fluorescence images of endothelial tube formation in the endothelial cell tube formation assay (FIG. 6C) after treated with low serum medium (1.5% FBS), 40 ng/mL VEGF, blank cryoMNs, and hMSCs-loaded cryoMNs (Scale bar: 100 μm);

FIGS. 17AA through 17AF show the viability of OVA-DCs inside the cryoMNs, and, specifically, FIGS. 17AA through 17AF show the gating strategies and the percentages of CD11c+CD86+ DCs and CD11c+MHCII+ DCs after pulsed with OVA and LPS;

FIG. 17B shows the live and dead staining of OVA-DCs after released from OVA-DCs loaded cryoMNs;

FIGS. 17CA through 17CC show the survival of OVA-DCs after short-term (i.e., 1 week in −80° C. ultralow freezer) and long-term (i.e., 1 and 3 months in liquid nitrogen) storage analyzing by flow cytometry;

FIG. 18 shows the optimization of OVA-DCs dosage through subcutaneous injection on a mouse model, wherein the tail snip blood is collected from second to sixth weeks and analyzed for OVA-specific IgG in plasma to evaluate systemic immune response (each group has two independent animals);

FIG. 19A shows the lymph node homing of released OVA-DCs from OVA-DCs loaded cryoMNs, the OVA-DCs being stained with CellTracker™ Green CMFDA Dye prior loaded inside the cryoMNs;

FIG. 19B shows green fluorescence positive DCs of mice lymph node analysis using flow cytometry and histology (three independent animals are analyzed; Scale bar: 100 μm); and

FIG. 20 shows the fabrication of the porous MN scaffolds based on various hydrogel formulations according to an embodiment of the present invention, including cryogelation of polyvinyl alcohol (PVA), cryogelation of gelatin (Gelatin), EDC/NHS (1-ethyl-3-(3-dimethylaminopropyl)carbodiimide/N-hydroxysuccinimide)-crosslinked gelatin (EDC/NHS-Gel), 1,4-Butanediol diglycidyl ether crosslinked hyaluronic acid (BDDE-HA), photocrosslinked gelatin methacryloyl (GelMA), photocrosslinked polyethylene glycol diacrylate (PEGDA), and Ca²⁺-crosslinked sodium alginate (Alginate) hydrogels.

The drawings herein are for reference purposes only and is not necessarily to scale.

### DETAILED DESCRIPTION OF THE INVENTION

The patent or application file contains at least one drawing executed in color. Copies of this patent or patent application publication with color drawing(s) will be provided by the Office upon request and payment of the necessary fee.

Unless otherwise noted, all measurements, weights, lengths etc. are in metric units, and all temperatures are in degrees Celsius. Furthermore, all percentages, ratios, etc. herein are by weight, unless specifically indicated otherwise. It is understood that unless otherwise specifically noted, the materials compounds, chemicals, etc. described herein are typically commodity items and/or industry-standard items available from a variety of suppliers and sources worldwide.

As used herein, the term “cryomicroneedle” (a.k.a., “cryoMN”) refers to a microneedle which is prepared and/or used while the microneedle is at a low temperature. Typically the cryomicroneedle is already loaded with a biological agent and frozen.

As used herein, the term “cryoprotectant” refers to any reagent which may protect a cell against sub-freezing temperatures, such as from about −196° C. to about −20° C. In the context of the present invention, the term “cryoprotectant” and “cryoprotective agent” may be used interchangeably.

As used herein the term “microneedle” or “MN” refers to a micro-sized needle. Such needles may have a height of from about 25 μm to about 2000 μm as described herein. They are generally in the form of an array or a patch, and may be made of various materials.

As used herein the term “MN scaffold” indicates the microneedle scaffold after it has been demolded but before the biological agent has been added thereto.

As used herein the term “MN master template” indicates the (positive) form used to create the (negative) mold. The MN master template is typically similar in shape as the desired MN scaffold, although it is recognized that a certain amount of shrinkage in various, or all, dimensions is typically seen in the fabrication process.

As used herein, the term “needle length” refers to the average length from the base of the microneedle and/or cryomicroneedle to the tip of the needles thereof.

As used herein the tem “needle base width” refers to the average longest width of the base of the microneedle and/or cryomicroneedle in the cross-section of the axial MN array.

An aspect of the present invention relates to a method for fabricating a cryomicroneedle, including: providing a microneedle scaffold including a plurality of pores; providing a suspension including a biological agent; loading the biological agent into the microneedle scaffold by immersing the microneedle scaffold in the suspension to form a loaded microneedle scaffold; and freezing the loaded microneedle scaffold to provide the cryomicroneedle.

Without intending to be bound by theory, it is believed that the microneedle scaffold may be made by any suitable material, to the extent that the material is safe to be used on a patient, suitable for providing the sufficient strength and may provide the desired porosity. The desired porosity refers to sufficient pore sizes to accommodate the biologics to be loaded into the microneedle scaffold. In some cases, a pore size of less than about 0.05 μm; a pore size of from about 0.05 μm to about 120 μm; or from about 0.1 μm to about 80 μm; or from about 0.2 μm to about 60 μm; 0.2 μm to about 50 μm; or from about 10 μm to about 50 μm may be preferred, depending on the size of the biologics to be loaded. For instance, mammalian cells have an average size of from about 10 μm to about 30 μm in suspension. As such, a pore size of from about 10 μm to about 50 μm may be preferred for loading mammalian cells. Likewise, bacteria and/or fungi generally have sizes as small as 0.2 μm, and thus a pore size of from about 0.2 μm to about 50 μm may be preferred. Pore sizes less than about 0.05 μm can be used for other biologics like proteins/peptides, nucleic acids, and cell extracts.

In an embodiment of the present invention, the microneedle scaffold contains a polymer. In this case, the microneedle scaffold may be made by a process including the steps of: providing a mold including a plurality of voids; providing a scaffold precursor solution; casting the scaffold precursor solution into the mold and filling the plurality of voids with the scaffold precursor solution; cross-linking the precursor to form a scaffold including a plurality of pores; lyophilizing the scaffold; demoulding the scaffold from the mold to form a microneedle scaffold; and optionally, the demolded microneedle scaffold is cross-linked again. A person of ordinary skill in the art should appreciate that the additional cross-linking step is to further strengthen the scaffold, and is thus optional.

In an embodiment herein, the scaffold contains a hydrogel, an aerogel, a biodegradable polymer, a metal, a bioceramic and a combination thereof; or a hydrogel. Without intending to be limited by theory, it is believed that these materials are capable of withstanding the fabrication process and also suitable for cell delivery process as described herein.

In an embodiment of the present invention, the scaffold precursor solution for fabricating the microneedle scaffold contains a precursor selected from the group of hyaluronic acid, agarose, alginic acid, chitosan, dextran, fibrin, gelatin/collagen, poly(ethylene glycol) (PEG), poly(ethylene oxide)(PEO), polyacrylamide (PAA), poly(vinyl alcohol) (PVA), polyglycolic acid (PGA), polylactic acid (PLA), stainless steel, titanium, aluminum, alumina, zirconia, calcium sulfate-based ceramics, calcium phosphate-based ceramics, a derivative thereof, and a combination thereof. As discussed above, a person of ordinary skill in the art should appreciate that other polymers may be used, to the extent that the polymers are safe and are capable to providing sufficient strength and desired porosity. It may be preferable for a material to provide tunable porosity, in that the material may be engineered to provide a wide range of pore sizes to load biologics of various sizes. In a specific example, microneedle scaffold is made of methacrylated hyaluronic acid (MeHA). In another specific example, the microneedle scaffold is made of photocrosslinked gelatin methacryloyl (GelMA).

For instance, where the microneedle scaffold is used to load biologics of smaller sizes, such as nucleic acids, vectors, proteins and cell extracts, it may be desirable to select a precursor which is capable of forming a microporous structure. In that case, the material strength required to form a macroporous structure may no longer be required.

It is also contemplated that additional functionalities may be introduced by selecting and/or modifying the material forming the microneedle scaffold. For instance, the freezing profile of a material may be altered to improve the cryopreservation outcome. The material may also be modified to prolong its thawing duration for ease of application. It is also contemplated that the material may be modified to manipulate its hydrophilicity to control the release rate of the loaded biologics. It is also possible to achieve controlled spatiotemporal release of the loaded biologics by introducing a composite structure to the microneedle scaffold.

In an embodiment of the present invention, the cross-linking of the scaffold precursor solution and/or cross-linking the demolded microneedle scaffold includes a step of exposing the precursor solution and/or the microneedle scaffold to a condition selected from the group consisting of light exposure, radiation exposure, copolymerization initiation, a thaw-freeze cycle, reduced temperature, ionic solution exposure, pH adjustment and a combination thereof. It should be understood that these optional steps are just for illustrative purposes. To the extent that the desired cross-linking is achieved, a person of ordinary skill in the art would be able to determine which step to perform or to exclude in the cross-linking step. Also, the skilled person would also be able to determine whether to perform additional steps for the cross-linking to provide the desired microneedle scaffold.

In another embodiment of the present invention, the microneedle scaffold may contain a material selected from the group consisting of a protein, a nucleic acid, a ceramic, a metal and a combination thereof. In some cases, the microneedle scaffold may be preferably made of protein and/or nucleic acid, due to their relative safety. Also, such materials may be metabolized within the body of a patient, and thus could be advantageous where the loaded biologics are to be delivered in a controlled manner over an extended period of time.

In an embodiment of the present invention, the microneedle scaffold has a needle length of from about 25 μm to about 2000 μm. A person of ordinary skill in the art should appreciate that the specific needle length may be adjusted, based on the specific delivery site of the biological agent carried by the cryomicroneedle.

In an embodiment of the present invention, the microneedle scaffold has a needle base width of from about 10 μm to about 750 μm. A person of ordinary skill in the art should appreciate that the specific needle base width may be adjusted, based on the specific delivery site of the biological agent carried by the cryomicroneedle.

In an embodiment of the present invention, the suspension further includes a cryoprotective agent selected from the group consisting of a cell membrane-penetrating cryoprotectant, a non-penetrating cryoprotectant, and a combination thereof. Without intending to be bound by theory, it is believed that the use of the cell membrane-penetrating cryoprotectant and/or the non-penetrating cryoprotectant enhances cell viability of the biological agent. In particular, the inventors of the present invention find that the combination of the cell membrane-penetrating cryoprotectant and the non-penetrating cryoprotectant produces an unexpected technical effect, in that the combination achieves satisfactory cell viability without needing to use a high amount of the cell membrane-penetrating cryoprotectant, which may in some cases be toxic to cells.

In an embodiment of the present invention, the cell membrane-penetrating cryoprotectants is selected from the group of dimethyl sulfoxide (DMSO), methanol, butanediol, proline glycerol, ethylene glycol, propylene glycol, diethylene glycol, triethylene glycol, glyceryl glucoside, formamide, acetamide, dimethylacetamide, trimethylamine, cell-penetrating zwitterionic cryoprotectant (e.g., betaine) and a combination thereof. In another embodiment, the non-penetrating cryoprotectant is selected from a non-permeable zwitterionic cryoprotectant, a polymeric cryoprotectant and a combination thereof. Without intending to be limited by theory, it is believed that some zwitterioinic and polymeric cryoprotectants (e.g., poly(ampholytes)) may mimic the properties of natural antifreeze proteins, and thus deliver cryoprotective functions.

In yet a further embodiment, the non-penetrating cryoprotectant is selected from the group of polyethylene glycol (PEG), polyvinyl pyrrolidone, polyvinyl alcohol (PVA), hydroxyethyl starch (HES), methyl cellulose (MC), carboxymethyl cellulose (CMC), dextran, polyproline, hyaluronic acid, alginic acid, carboxylated poly-L-lysine, a poly(ampholyte) and a combination thereof. A person of ordinary skill in the art should appreciate that other suitable selections for the cell membrane-penetrating cryoprotectants and the non-penetrating cryoprotectants are also within the scope of the present invention, to the extent that such selections equally achieve enhanced cell viability. In some cases, it may be particularly desirable to use a cell membrane-penetrating cryoprotectant of low toxicity, such as betaine.

Prior to the present invention, cell membrane-penetrating cryoprotectants are generally used in high amounts (e.g., at least 10% by volume). However, it has been surprisingly found that the present invention reduces the amount of a cell membrane-penetrating cryoprotectant required for maintaining cell viability. In an embodiment of the present invention, the amount of the cell membrane-penetrating cryoprotectant is less than about 10% by volume; or less than about 5% by volume; or less than about 3% by volume.

In an embodiment of the present invention, the biological agent is selected from the group consisting of a cell organoid, a cell aggregate, a cell, a bacterium, a virus, a protein/peptide, a nucleic acid/DNA/RNA, a cell extract or component, a cell-mimicking particle, a vector, and a combination thereof. It should be understood that these specific selections are for illustrative purposes. Any biological agent which may be loaded into the cryomicroneedle of the present invention and transdermally delivered thereby may fall within the scope of the present invention.

Another aspect of the present invention relates to a cryomicroneedle prepared according to the method of the present invention. Such a cryomicroneedle may be loaded with a biological agent of interest and transdermally delivers the agent to a desired location on a patient. Such a cryomicroneedle may satisfactorily preserve the viability, activity and function of the biological agent to be delivered, and is easy to prepare, to store as well as to use.

FIG. 1 illustrates the fabrication of a cryomicroneedle according to an embodiment of the present invention. Specifically, a sponge-like, porous MN scaffold is prepared such that it allows the loading of cells by a one-step dipping process. The porous MN scaffold is made of crosslinked MeHA through lyophilization. Cells suspended in an embodiment of an optimized cryopreservation formula are then loaded into the porous MN scaffold during the dipping process, driven by the capillary force.^{[5]} The loaded MN scaffold is directly frozen and stored in liquid nitrogen/−80° C. freezer until usage.

Fabrication and Characterization of porous MN scaffold

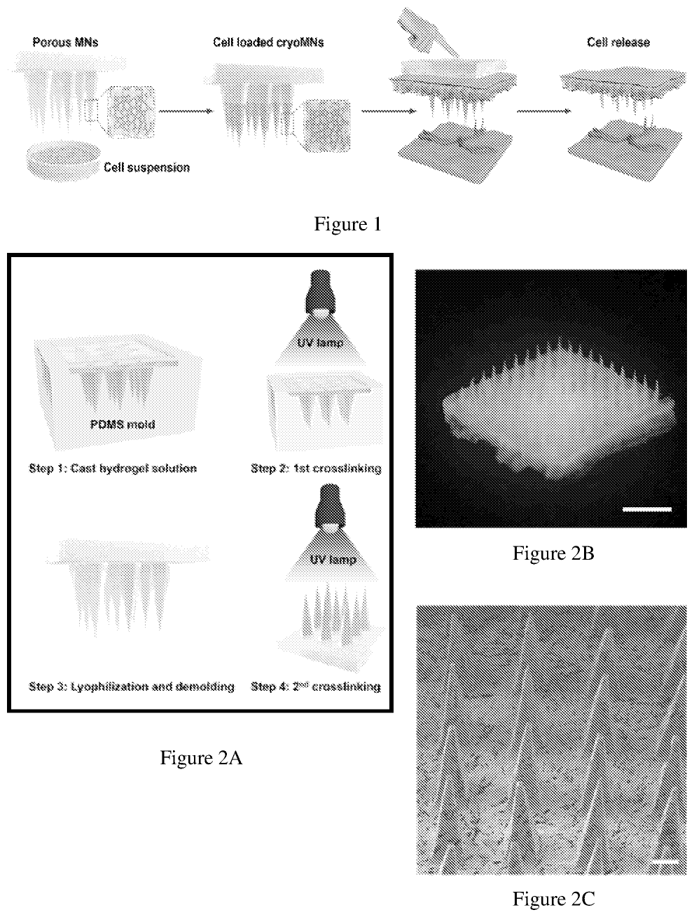

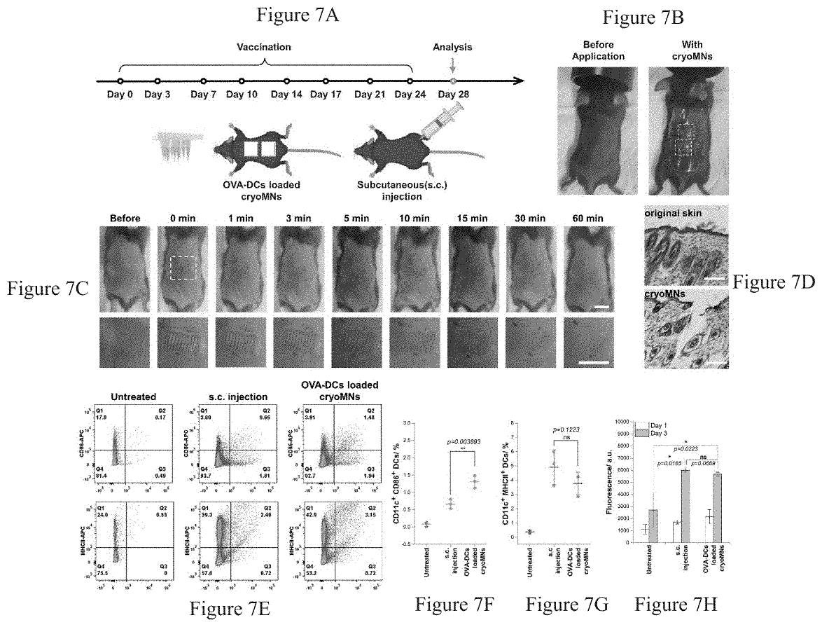

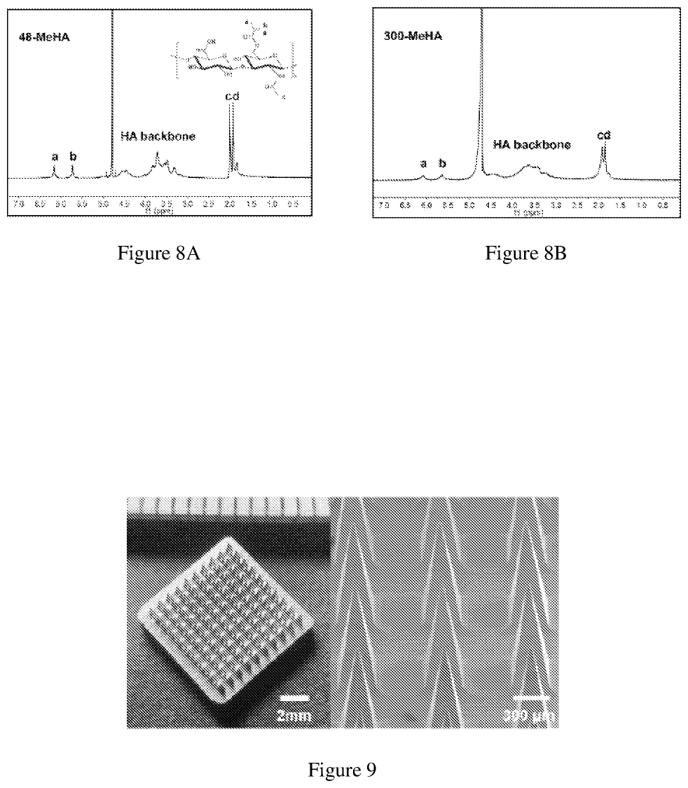

Hyaluronic acid (HA) with different sizes (Mw=48 kDa and 300 kDa) is modified with methacrylic anhydride to derive the photocrosslinkable 48-MeHA and 300-MeHA. The degrees of substitution, evaluated by 1H NMR, are 93.6% and 70.3% for 48-MeHA and 300-MeHA respectively, as shown in FIG. 7. The porous MN structure is fabricated using a micromolding method with MeHA and porogen, as shown in FIG. 2A. The porogen in this embodiment is ice, which introduces the micropores in the MN structure.^{[6]} The MeHA aqueous solution is cast into the negative polydimethylsiloxane (PDMS) mold and then centrifuged to fill the void. After cross-linking under the exposure of UV light, MeHA MNs are lyophilized and peeled off to derive the porous MN scaffold. FIG. 2B and FIG. 2C show that the final MN scaffold shows a morphology similar the original MN master template. The MN master template (typically made of stainless steel) is first used to fabricate a (negative) mold. Including a plurality of voids. The (negative) mold is then cast by filling the scaffold precursor solution into the (plurality of voids in) the mold, cross-linking the scaffold precursor solution to generate a scaffold with a plurality of pores, lyophilizing the scaffold, and demoulding the scaffold to generate the MN scaffold made from the desired materials.

The MN scaffold is theoretically the same geometry as the MN master template. In an example herein, the stainless-steel master MN master template has a height of 1200 μm and a base width of 300 μm, as shown in FIG. 9. However, the height and base for porous MN scaffolds made from this MN master template and containing 48-MeHA are 700 μm and 270 μm, respectively. For MN scaffolds containing 300-MeHA, the respective height and width are 620 μm and 320 μm. The shrinking of the MN dimensions has been observed in many previous studies, which is due to the shrinkage of PDMS and polymeric matrix.^{[7, 8]} The MNs may be loaded with their desired cargoes without the aid of further specialized equipment.

In an embodiment herein, the MN scaffold possess a needle height (as measured perpendicular to the base and from the base to the tip of the needle) of from about 25 μm to about 2000 μm; or from about 50 μm to about 1800 μm; or form about 100 μm to about 1500 μm.

In an embodiment herein, the MN scaffold possess a needle width (as measured at the base or widest part of the needle) of from about 10 μm to about 750 μm; or from about 15 μm to about 700 μm; or form about 20 μm to about 600 μm.

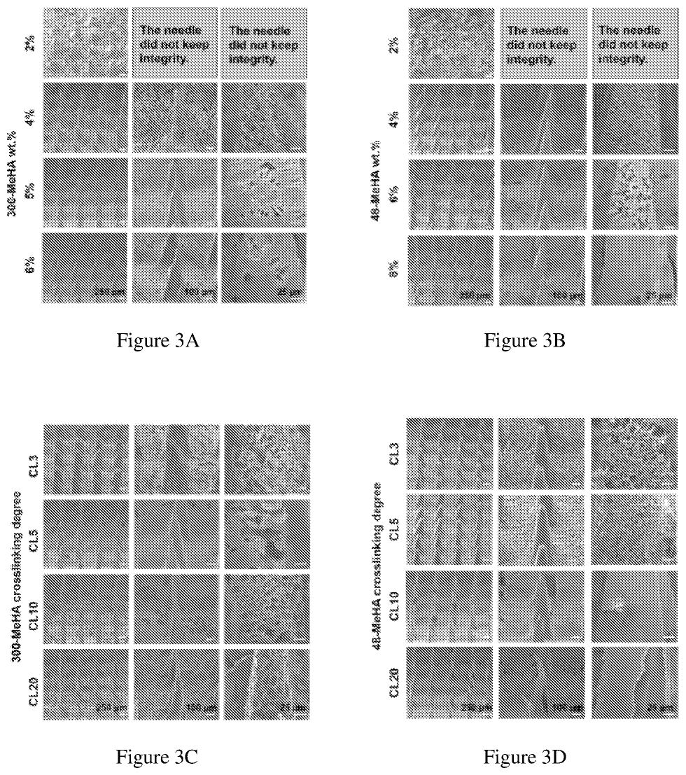

The porous structure of the MNs may be tunable by adjusting the MeHA concentration and cross-linking time in the fabrication process. The morphology of MN scaffolds made with different MeHA concentrations (2, 4, 5, 6 wt. % for 300-MeHA; 2, 4, 6, 8 wt. % for 48-MeHA) under the same cross-linking time (5 mins) is examined. As shown in FIG. 3A and FIG. 3B, both 48-MeHA and 300-MeHA MNs gain the porous structure when the concentration of polymer is 4%. A lower concentration does not provide a stable MN structure while the higher concentrations significantly decrease the porosity.^{[5, 9]} The cross-linking time is another important parameter. FIG. 3C and FIG. 3D show the optimized MeHA concentration (i.e. 4%) with the UV cross-linking times of 3 min (CL3), 5 min (CL5), 10 min (CL10), and 20 min (CL20). In general, the longer the UV exposure time, the lower the porosity.^{[10]} Specifically, when the cross-linking is 10 min or longer, a shell structure forms instead of the porous structure. This shell structure is not preferred as it reduces the cell loading.

Nevertheless, CL3 and CL5 of 48-MeHA (i.e. CL3-48-MeHA and CL5-48-MeHA) and CL3 of 300-MeHA (i.e. CL3-300-MeHA) are identified as preferred due to their intact MN structure and observable porosity. Their porosity and dimension are further studied through SEM images using ImageJ. CL3-48-MeHA and CL5-48-MeHA MNs have the average MN height of 697.1±21.0 μm and 682.9±10.7 μm, respectively. CL3-300-MeHA MN is slightly shorter at 616.4±32.8 μm. The average pore sizes for CL3-48-MeHA, CL5-48-MeHA, and CL3-300-MeHA are 81.0±36.8 μm, 56.6±22.8 μm, and 54.2±23.9 μm, respectively. The pore size matches well with the mammalian cells that are usually 10-30 μm in suspension. Out of these three embodiments, CL5-48-MeHA is used as the representative for the following studies.

Optimization of Cryoprotective Medium

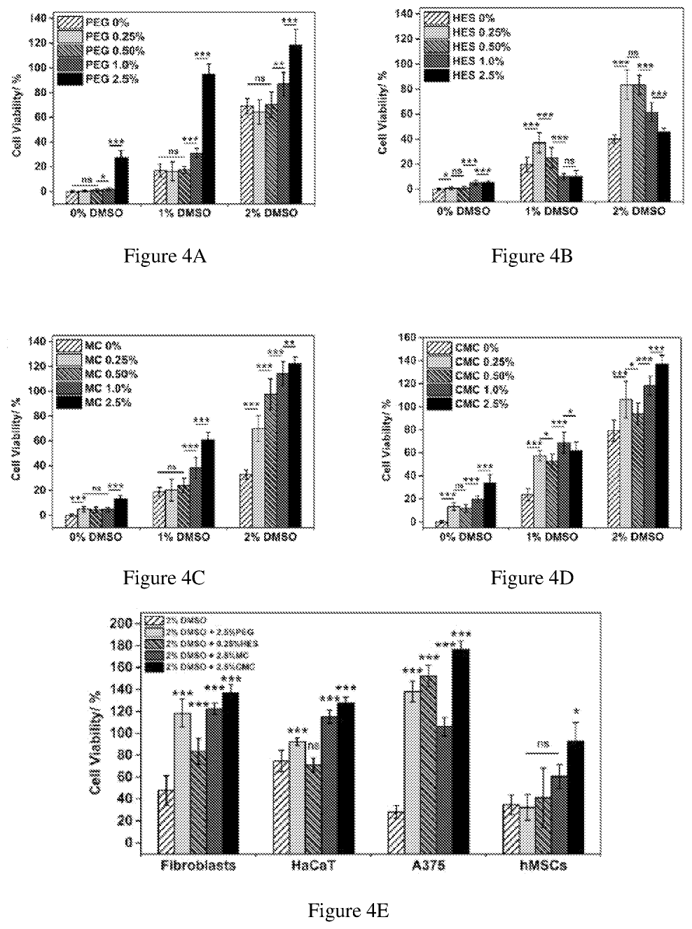

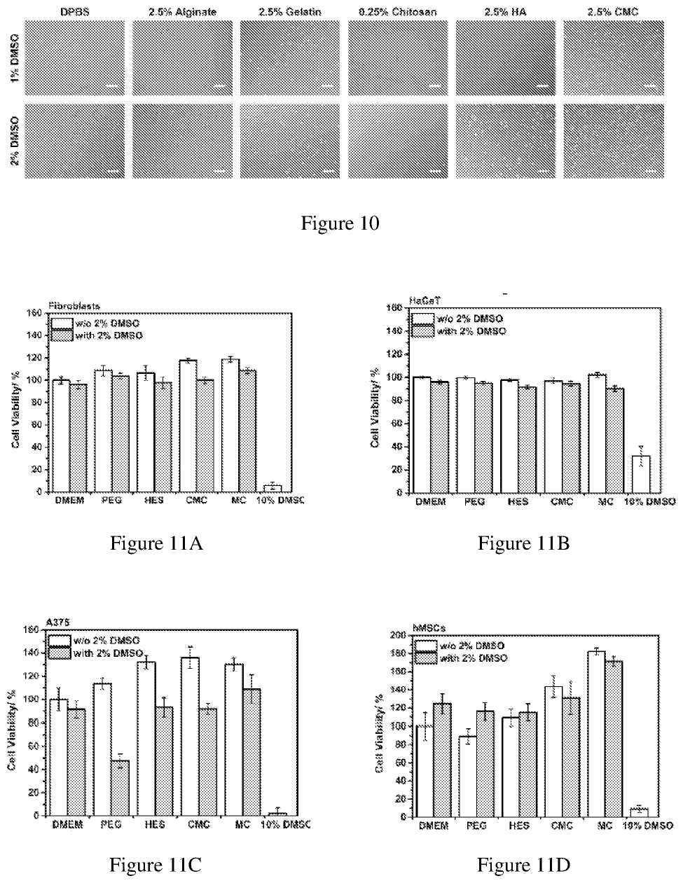

Dimethyl sulfoxide (DMSO) is the most popular cryoprotective agent by binding with water molecules to prevent the ice crystallization and cell damages.^{[11]} However, it is toxic to the cells with the standard concentration in cryopreservation (i.e. 10%, v/v). During the intradermal delivery of the therapeutic cells, the high dosage of DMSO in the cryoprotective solution might bring unwanted side effects to surrounding skin cells. To minimize the concentration of DMSO in the cryoprotective medium, the hydrogel-forming polymers are incorporated into the cryoprotective solution with a relatively lower concentration of DMSO. Hydrogel-forming polymers have been featured with good biocompatibility and good affinity with water molecule. Several polymers such as polyethylene glycol (PEG), and hydroxyethyl starch (HES) have been applied in the cryopreservation for red blood cells^{[12]}, stem cells^{[13]}, and other mammalian cells^{[14]}. The present invention explores eight common hydrogel polymers for their protective capacity to cryopreserve cells, including PEG, HES, methylcellulose (MC), sodium carboxymethyl cellulose (CMC), HA, chitosan, gelatin, and alginate. Specifically, the hydrogel polymer is mixed with 1% of DMSO under different concentrations for cryopreserving cells, using human dermal fibroblasts as the model cell. After 3-day preservation at −80° C., cells are thawed for the assessment of cell viability. In general, the cryogenic media containing PEG, HES, MC and CMC ensure the cell survival (shown by FIGS. 4A-D). Unfortunately, media containing HA, chitosan, gelatin, and alginate do not provide comparable results (shown by FIG. 10).

The concentrations of PEG, HES, MC and CMC are further screened as it can alter the ice crystallization and cell dehydration, and finally affect the cryoprotective functions. The polymer concentration is tuned from 0, 0.25, 0.5, 1 to 2.5 wt. % in Dulbecco's phosphate-buffered saline (DPBS) solution, supplemented with DMSO ranging from 0%, 1% to 2% (v/v) (FIGS. 4A-D). The cryogenic solution, which only contains hydrogel polymer ingredients, do not show sufficient protection of cells in cryopreservation with cell viability all lower than 40%. However, with only 1% of DMSO, PEG achieves comparable cell viability to a standard 10% DMSO formula. With 2% DMSO, all cryogenic solutions of four hydrogel polymers provide similar or even higher cell viability than 10% of DMSO. For formulations containing PEG, MC, and CMC, the highest concentration of these hydrogel polymers (2.5 wt. %) produce the highest cell viability. The only exception is HES-containing medium that provides the highest cell viability at a lower concentration of 0.25 wt. %. As such, by using a combination of a hydrogel polymer and low concentration of DMSO, an unexpected synergistic technical effect is produced that preserves cell viability while keeping the toxicity from DMSO as low as possible.

The preservation capability of these four preferred formulations (i.e. 2.5 wt. % PEG, 2.5 wt. % MC, 2.5 wt. % CMC, 0.25 wt. % of HES, all added with 2.0% DMSO) are further confirmed on other three types of human cells, including human keratinocytes (HaCaT), human malignant melanoma cell line (A375), and human bone marrow derived MSCs (hMSCs). As shown in FIG. 4E, all types of cells maintain comparable if not higher viabilities in these cryogenic formulations to those in the medium with 2% DMSO. More importantly, the cryogenic solution featured with PEG, HES, MC, and CMC do not induce any significant toxicity compared with medium with 10% DMSO (as shown by FIG. 11). A formulation containing 2.5 wt. % of CMC and 2% DMSO is finally selected as the representative cryogenic solution for the subsequent cell loading.

Fabrication of Cell-Containing cryoMNs from a Porous MN Scaffold

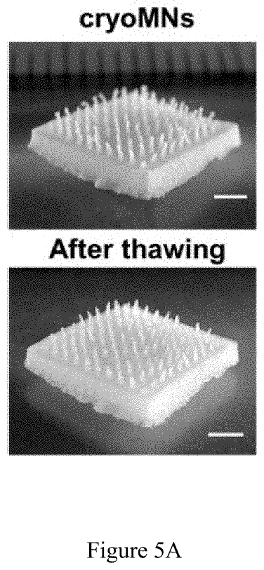

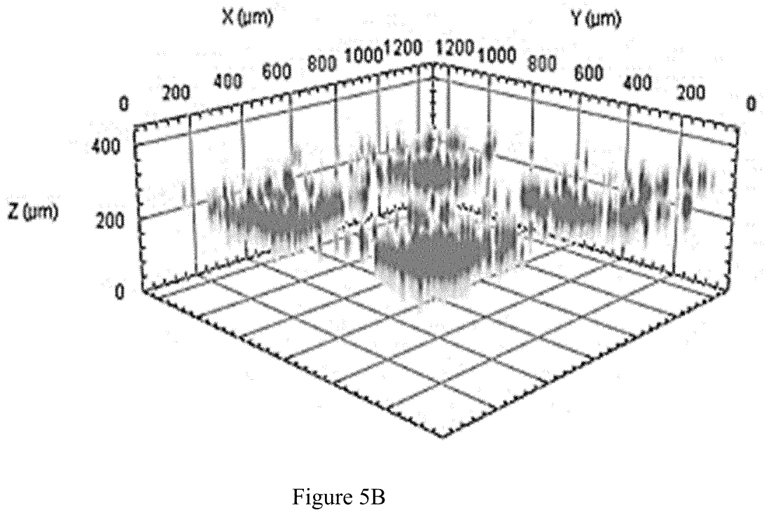

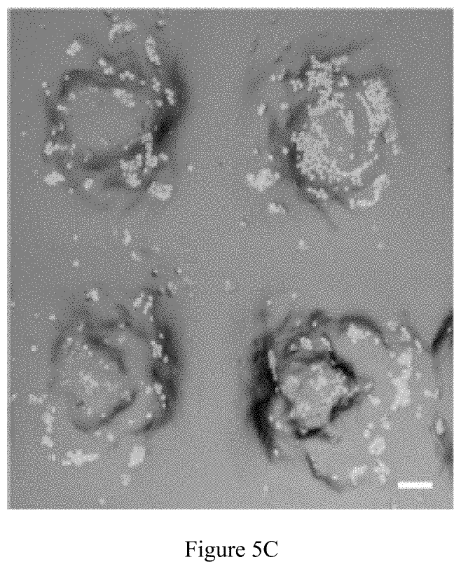

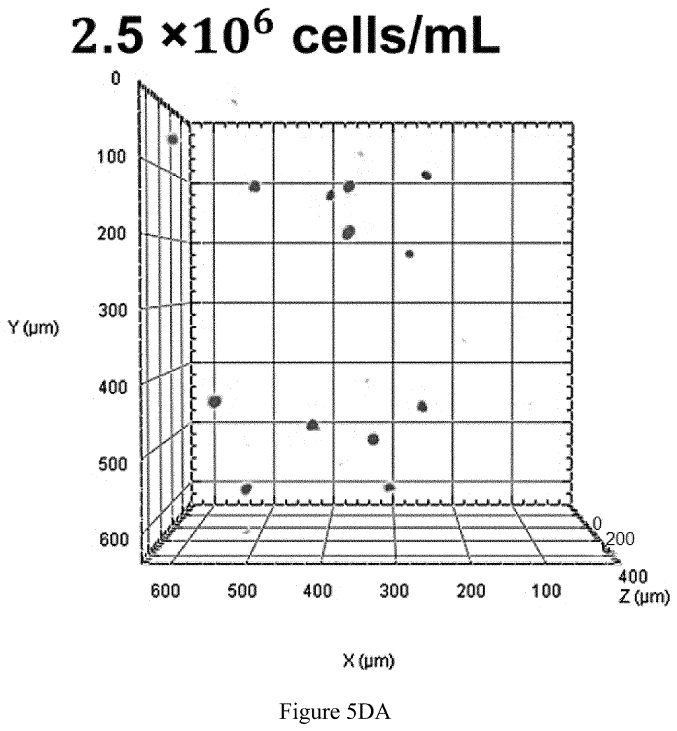

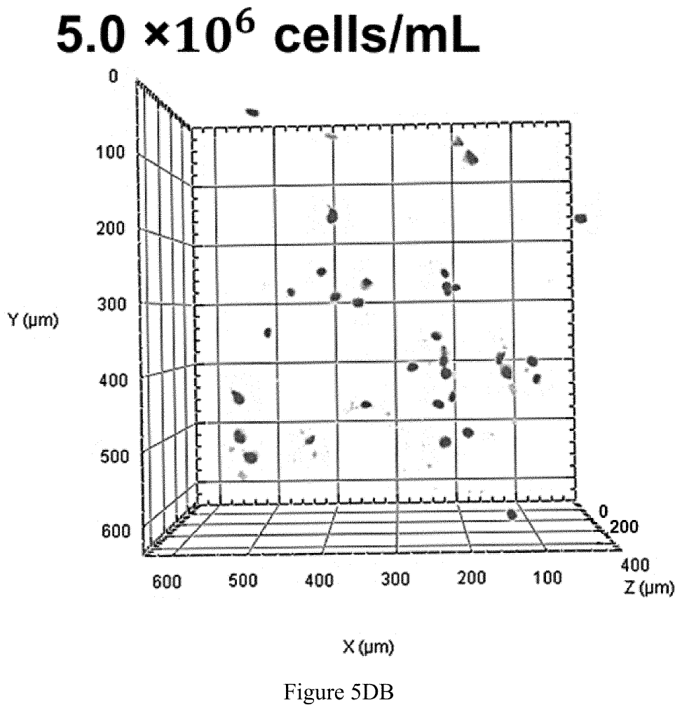

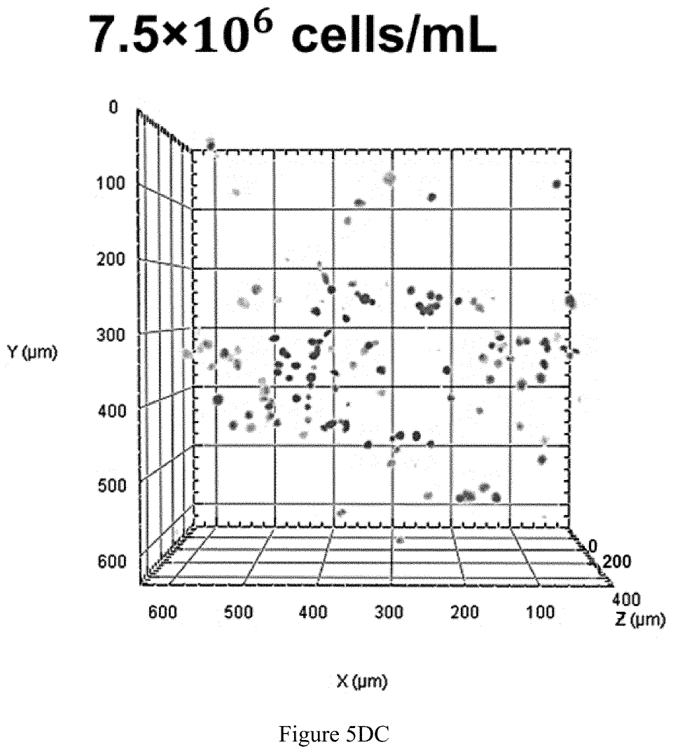

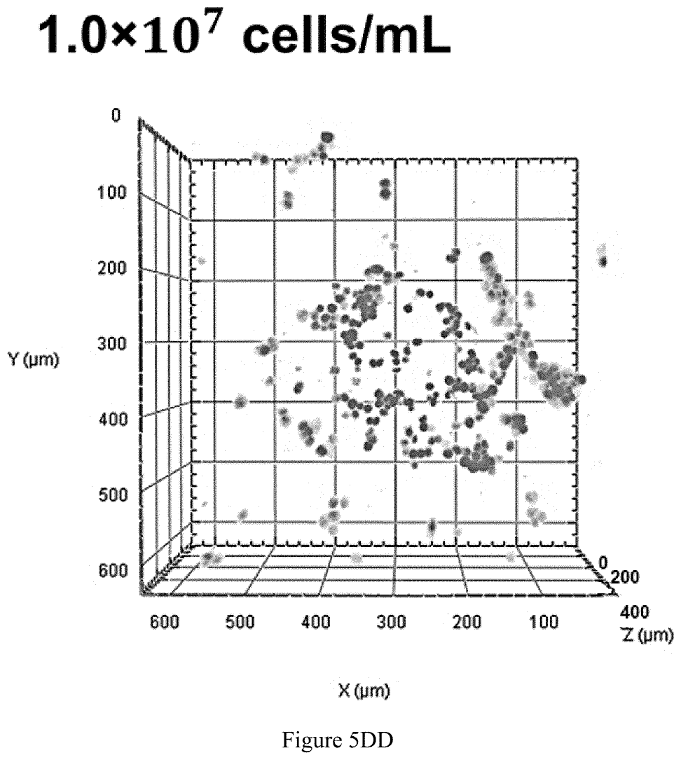

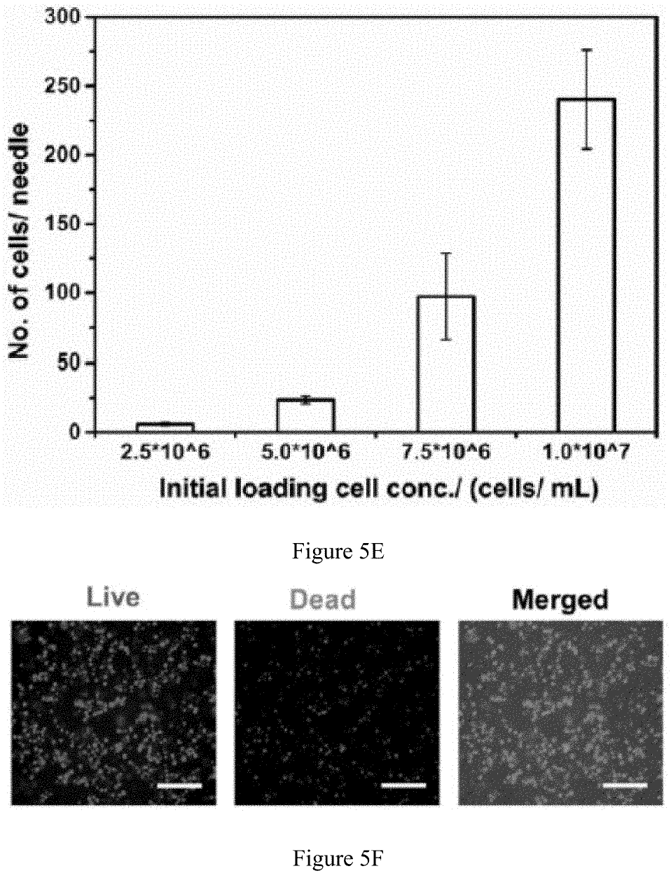

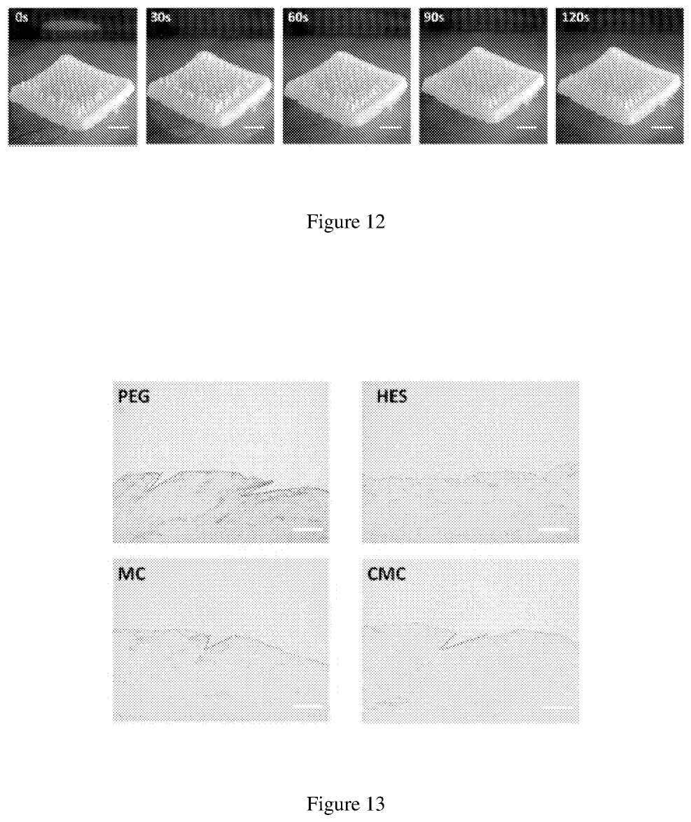

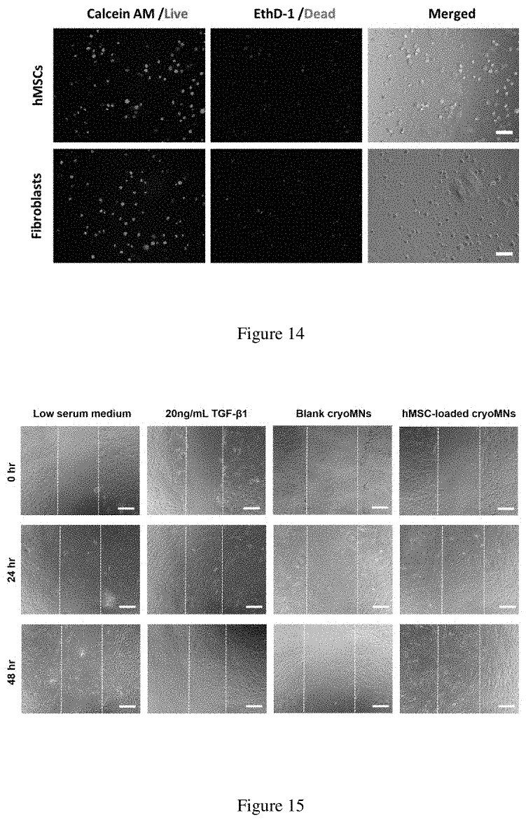

The porous MN scaffold (CL5-48-MeHA) is immersed in the cryogenic medium (2.5 wt. % CMC, 2% v/v DMSO in DPBS buffer) containing cells for 1 min (as shown by FIG. 1). Later, the cell-loaded cryoMN patch is frozen using the cryopreservation box. After overnight freezing, the patch is stored at −80° C. for short-term or in liquid nitrogen for longer storage (>7 days). As an example, A375 cells are loaded into the cryoMNs as described (as shows by FIG. 5A). When the frozen cryoMNs are exposed to room temperature (24° C.), they slowly melt and totally thaws after 2 mins (as shown by FIG. 12), accompanied by the temporary formation of frost on the needle tips. Throughout the thawing process, the cryoMNs keeps all the cells in the scaffold and maintains the MN morphology. Later, the cell-loaded cryoMNs are inserted into the agarose hydrogel (as shown by part FIG. 5B). The cells are pre-stained with CellTracker™ (green dye) for easy visualization. As shown in FIG. 5B and FIG. 5C of, the cryoMNs easily penetrate the agarose gel and deliver the stained A375 cells inside the agarose gel with a depth of around 200 μm. The mechanical strength of cryoMNs is also validated on ex vivo porcine skin, where the cryoMNs could be easily inserted into pig ear skin and prove the approximate penetration depth of 450 μm from the histological images (FIG. 13). The number of loaded cells inside the cryoMNs can be tuned to the initial cell density in the cell suspension solution. When the cell concentration is raised from 2.5×10\hat{ }6 to 5.0×10\hat{ }6, 7.5×10\hat{ }6, and 1.0×10\hat{ }7 cells/mL in the cryogenic medium, the numbers of cells accommodated in each needle increases accordingly from about 10 cells/needle up to 240 cells/needle (as shown by FIGS. 5DA through 5DD and FIG. 5E). Finally, the good viability of the released A375 cells is confirmed using the live/dead assay (as shown by FIG. 5F). Similarly, both hMSCs and fibroblasts can be loaded and cryopreserved in the cryoMNs with high cell viability (as shown by FIG. 14).

In Vitro Effects from cryoMN Delivered hMSCs

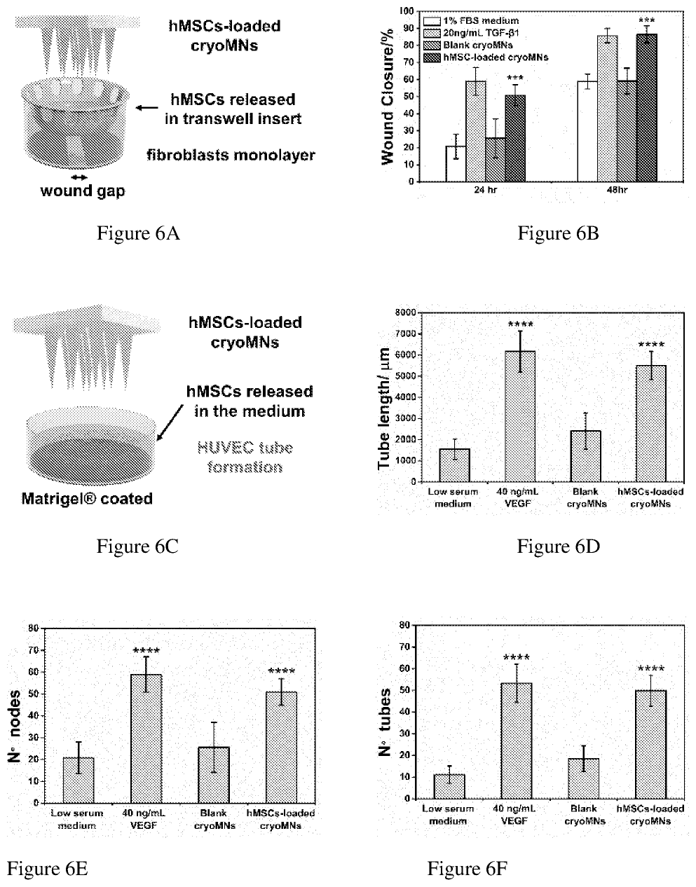

The scratch assay is performed to examine the migration capability of dermal fibroblasts in response to hMSCs delivered using cryoMNs (as shown by part A of FIG. 6). The migration and proliferation of fibroblasts into the wounded area at 24 hrs and 48 hrs post the treatment are examined under four conditions: low serum group (1% FBS instead of 5% FBS in other groups), positive control group (20 ng/mL TGF-β1), blank cryoMNs group, and hMSCs-loaded cryoMNs. As shown in FIG. 6B and FIG. 15, treatment with hMSCs-cryoMNs significantly promote the migration of fibroblasts. The closure is 50.8±6.1% at 24 hrs and 86.5±5.0% at 48 hrs compared while the value is 25.5±11.4% at 24 hrs and 59.1±7.6% at 48 hrs for the group treated with the blank cryoMNs. This result is comparable to that from the positive control.

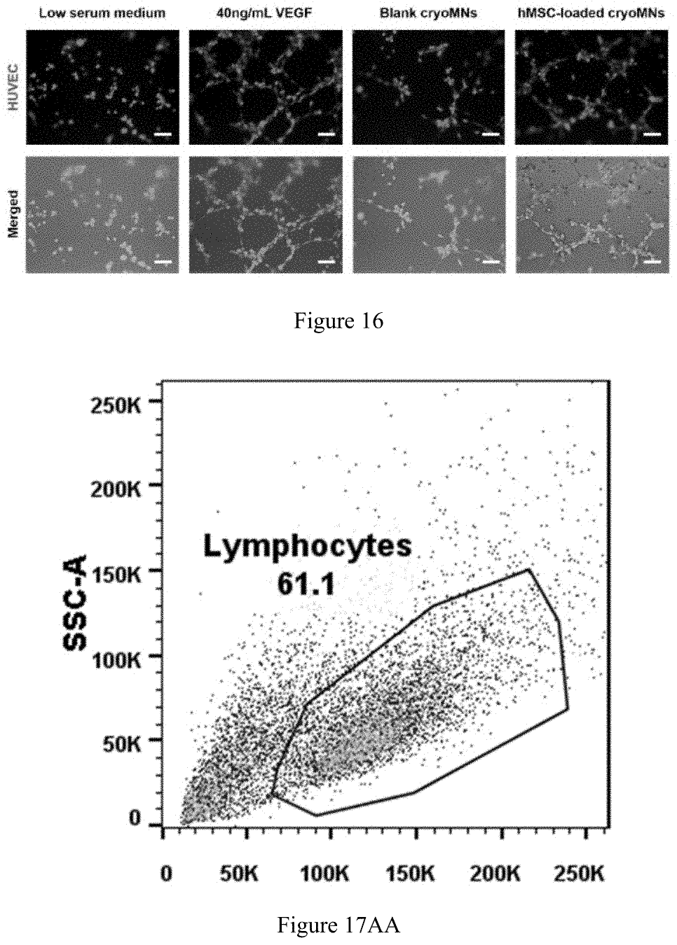

A tube formation assay (as shown FIG. 6C) is carried out and four conditions are examined including the low serum (1.5% FBS instead of 5% FBS in other groups), positive control (40 ng/mL VEGF), blank cryoMNs, and hMSCs-loaded cryoMNs. 8 hrs post the treatment, the tube-like network is clearly seen (as shown by FIG. 16). For the group treated with hMSCs-loaded cryoMNs, there is 2.3-time increase of total tube length to that treated with blank cryoMNs (as shown by FIG. 6D). The treatment with hMSCs-loaded cryoMNs increases the number of tubes and branching nodes for 2.7 times and 2.0 times as well, respectively to that of blank cryoMNs treatment (as shown by FIG. 6E and FIG. 6F).

In Vivo Delivery of Dendritic Cell Vaccines Using cryoMNs

Autologous cell therapy utilizes cells originating from a subject, such as an individuals and/or a patient and returns the extracted cells back to the patients themselves. A biological sample (e.g., a cell) is collected from a subject, engineering the biological sample into a biological agent (e.g., therapeutic cells and/or cells with some therapeutic efficacy), and multiplying the biological agent ex vivo before reintroducing to patients, in which minimizing manipulation largely preserves the cell qualities. Returning the biological agent (e.g., the therapeutic agent based on the extracted biological sample) may be conducted by, for example, affixing a patch containing the cryomicroneedles thereupon or formed thereupon to the epidermis of the subject so as to inject the biological agent into the subject. Without intending to be limited by theory it is believed that this embodiment of the invention may adopt a mild dip-loading procedure without the need for an on-site MN master template and centrifuge to prepare cell-laden cryoMNs from pre-prepared MN scaffolds, and thus allows on-site preparation and convenient intradermal delivery of autologous cells.

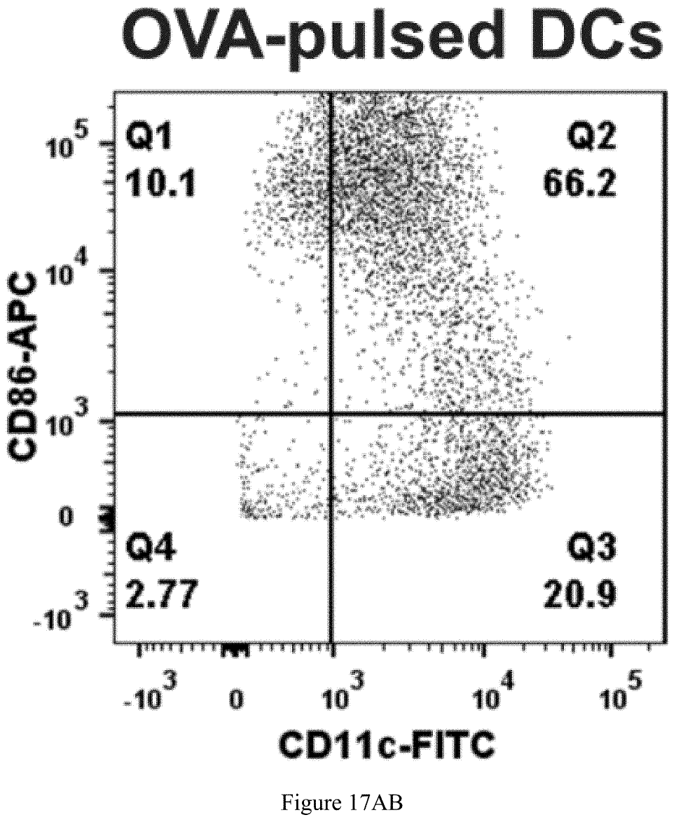

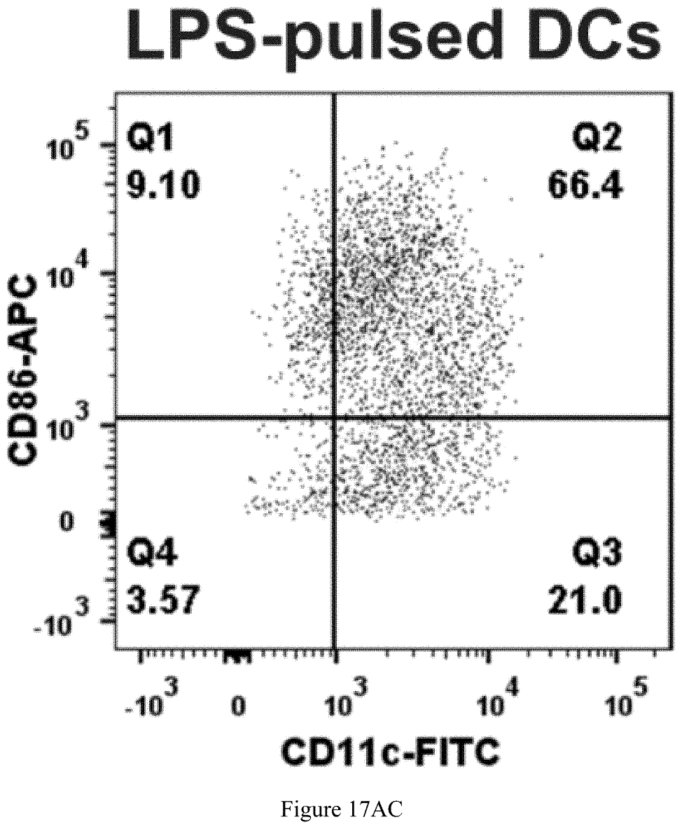

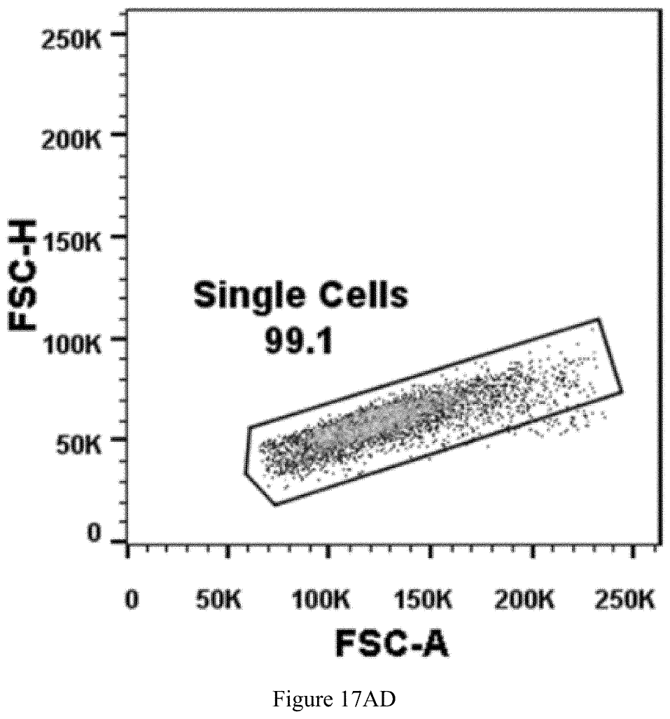

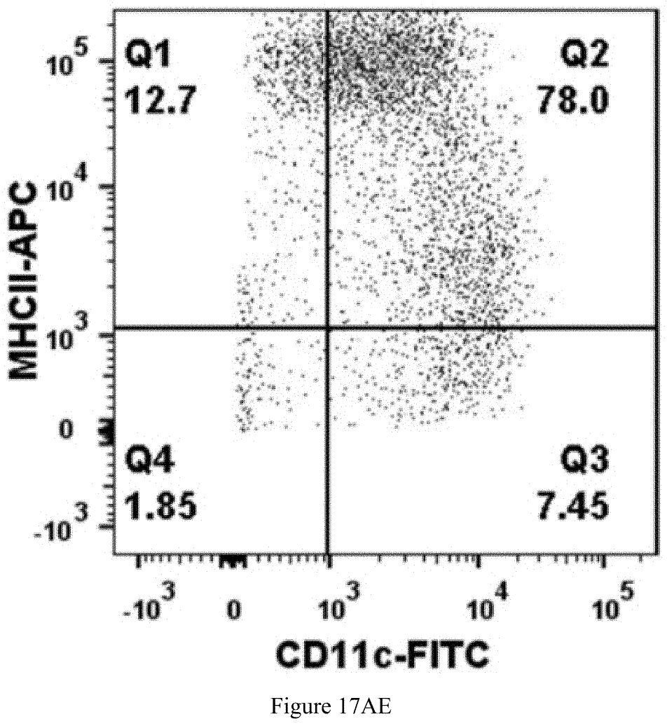

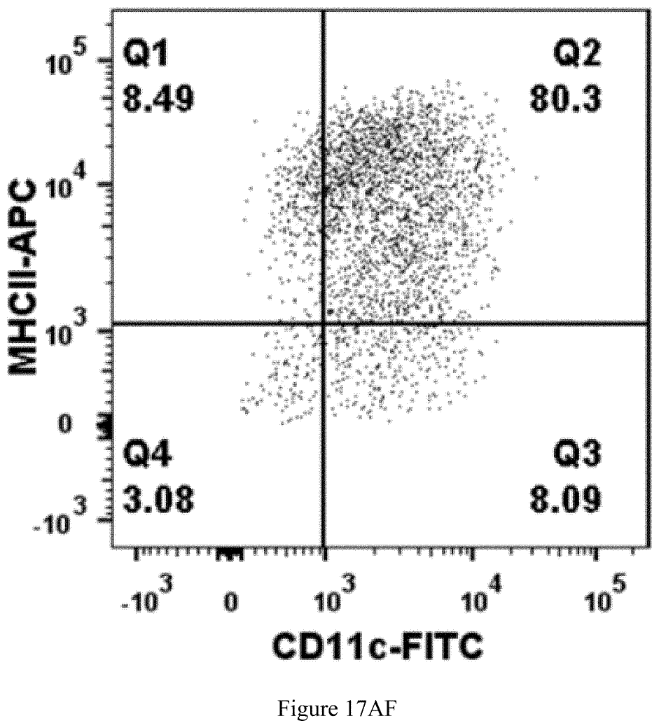

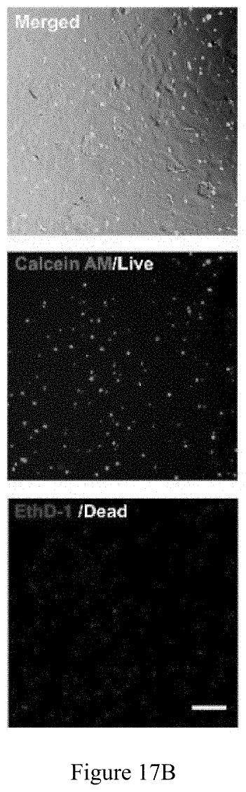

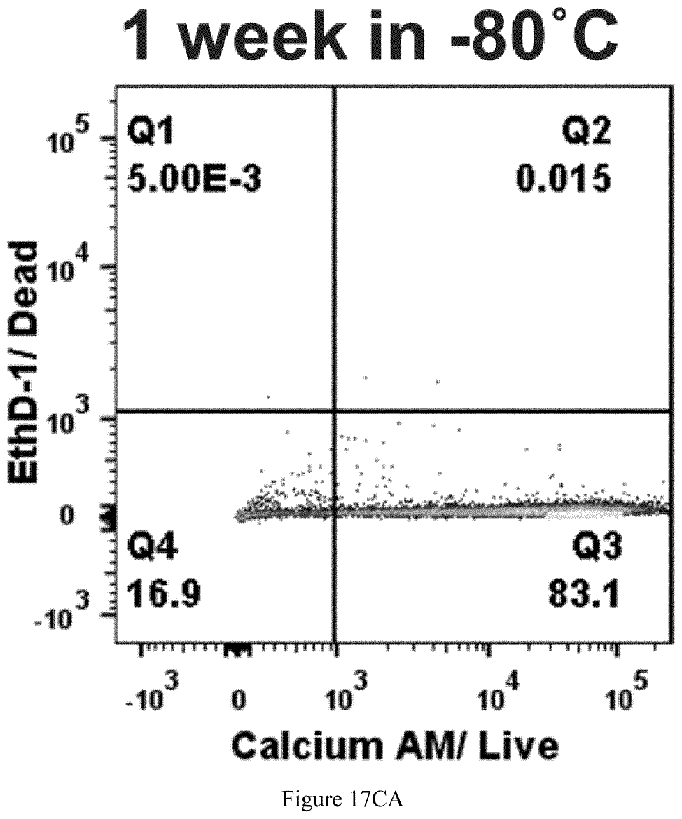

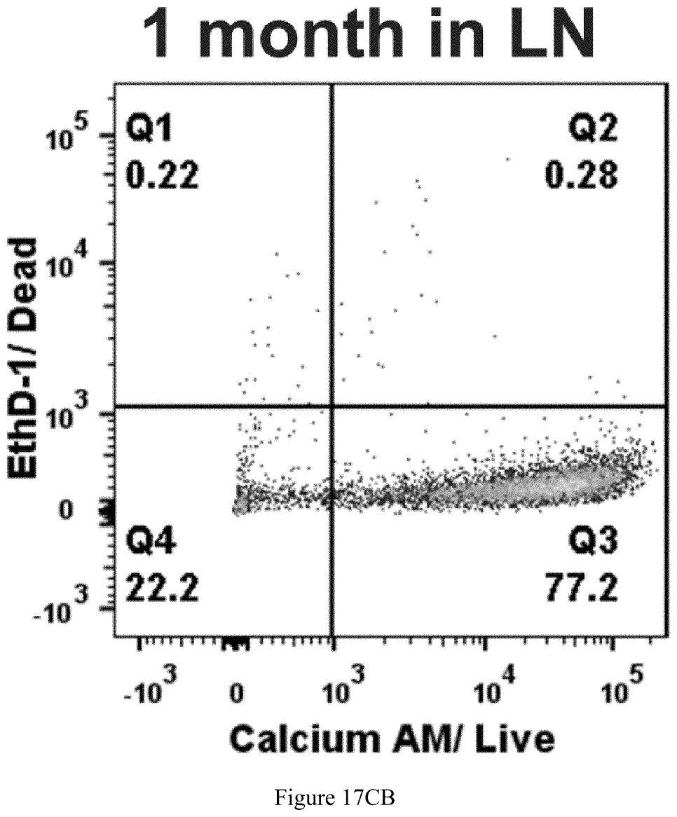

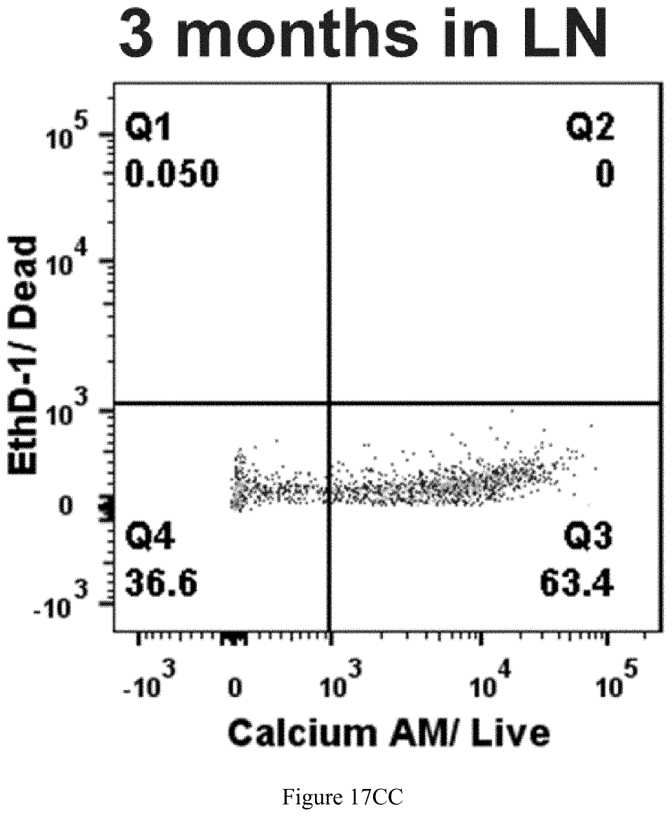

In an embodiment herein, dendritic cells (DCs) are potent antigen-presenting cells (APCs) to capture and present antigens to T lymphocytes for long-lasting and specific immune memory. In clinical, autologous dendritic cells are collected and exposed to tumor antigens to develop the DC vaccine, which is infused back to the patients to stimulate immune response and induce anti-tumor effects. In this study, we develop the antigen-pulsed DCs with a similar protocol reported in our previous work¹⁵, in which ovalbumin (OVA) is used as the model antigen. The OVA-pulsed DCs (OVA-DCs) are analyzed for their expression of surface markers of mature and active DCs (i.e., CD11c, CD86, and MHCII), and shows a comparable level with the positive control that treated with lipopolysaccharide (LPS) (FIGS. 17AA through 17AF). After loaded into cryoMNs (OVA-DC loaded cryoMNs), the OVA-DCs keep high viability after short-term (83.1%, 1 week in −80° C.) and long-term (77.2% for 1-month storage in liquid nitrogen; 63.4% for 3-month storage in liquid nitrogen) storage (FIG. 17B and FIGS. 17CA through 17CC).

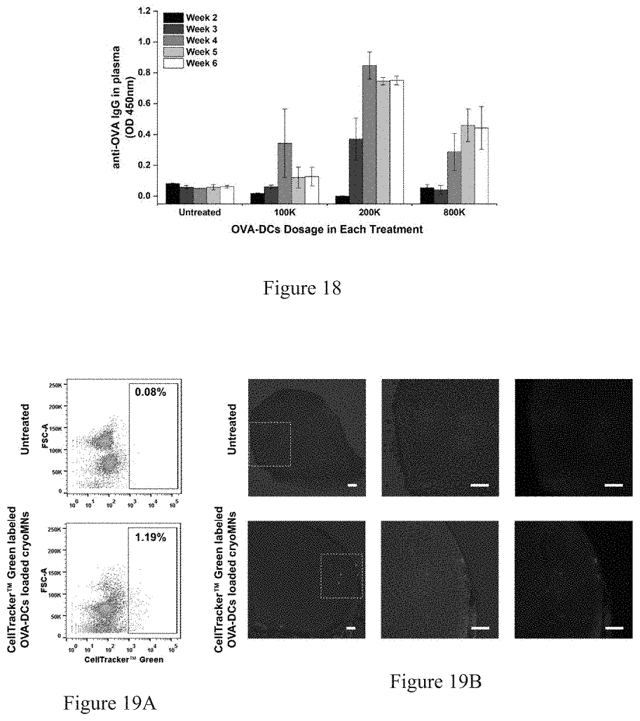

To investigate the optimal dosage and treatment times of OVA-DCs vaccination, the mice receive subcutaneous (s.c.) vaccination twice a week with different dosages of 0, 1, 2, 8×10\hat{ }5 OVA-DCs in each treatment. It is found that the levels of OVA-specific antibodies significantly increase at the third week and saturate after eight times of treatments (FIG. 18). In FIG. 18, the dosage at 2×10\hat{ }5 OVA-DCs shows the better antigen-specific immune response and is therefore chosen as the therapeutic dosage in tumor vaccination. OVA-DCs loaded cryoMNs delivering 8 doses of 2×10\hat{ }5 OVA-DCs (two patches each dose, each patch containing 1×10\hat{ }5 OVA-DCs) over four weeks to vaccinate the mice, which is compared with conventional s.c. injection (FIGS. 17AA through 17AF and FIG. 17B). After the application of cryoMNs, a clear micropattern showed up on the mouse skin and this micropattern visually recovered within 1 hour (FIG. 7C). And as shown in FIG. 7D, the cryoMNs can penetrate mouse skin up to ˜200 μm in the dermis area after histological evaluation. With one time administration of prestained OVA-DCs loaded cryoMNs, it is observable that labeled DCs are present in the draining lymph nodes (FIG. 19). And after 8 doses of vaccination, mice vaccinated using OVA-DCs cryoMNs have a higher percentage of CD11c+CD86+ DCs and a comparable percentage of CD11c+MHCII+ DCs in excised draining lymph nodes, which is compared with a conventional s.c. injection route (part E, F, and G of FIG. 7). The splenocytes are collected from vaccinated mice and restimulated with OVA, in which cryoMNs vaccinated mice have shown a similar proliferation with s.c. route vaccinated mice (FIG. 7H). This data indicates that the OVA-DCs loaded cryoMNs can be applied for delivery of DC vaccination and induce a specific immune response. Accordingly, it is believed that the cryomicroneedle herein may be employed in, for example, an autologous cell therapy method and/or a vaccination method of, for example, a mammal; or a mouse; or a human.

Fabrication of Porous MN Scaffolds from Various Hydrogel Formulations

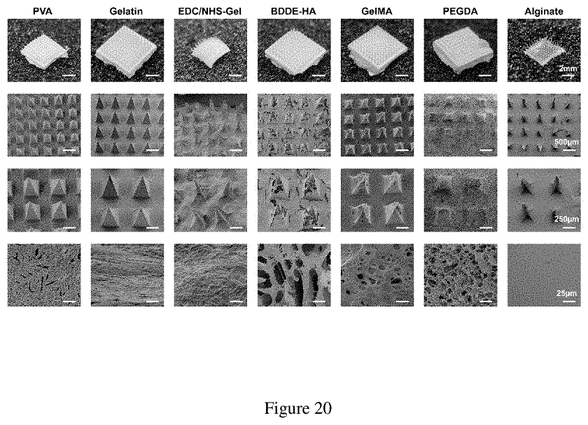

As shown by FIG. 20, porous MN scaffolds may be prepared by cryogelation of various hydrogel formulations according to an embodiment to the present invention. Suitable hydrogels include cryogelation of polyvinyl alcohol (PVA), cryogelation of gelatin (Gelatin), EDC/NHS (1-ethyl-3-(3-dimethylaminopropyl)carbodiimide/N-hydroxysuccinimide)-crosslinked gelatin (EDC/NHS-Gel), 1,4-Butanediol diglycidyl ether crosslinked hyaluronic acid (BDDE-HA), photocrosslinked gelatin methacryloyl (GelMA), photocrosslinked polyethylene glycol diacrylate (PEGDA), and Ca′-crosslinked sodium alginate (Alginate) hydrogels. Based on this, it is believed that a person of ordinary skill in the art may determine the specific selection of the hydrogels based on factors such as the desired porosity and strength of the MN scaffold.

Discussion

The present invention relates to the development of a porous MN scaffold for the fabrication of cell-loaded cryoMNs. The sponge-like structure of the porous MN scaffold allows the absorption of the cell suspension and the maintenance of MN morphology. This would facilitate the potential large-scale fabrication and distribution of the porous MN scaffold that can be loaded with cells and frozen to cryoMNs in situ by the nurse and clinicians for the intradermal injection of cells.

In the examples, the sponge-like MN scaffold is made through two-step UV cross-linking (as shown by FIG. 2). The first cross-linking in the PDMS mold is to achieve the proper pore size to accommodate the therapeutic cells (as shown by FIG. 3). The second cross-linking is carried on MeHAMNs peeled off from the mold, which is to strength minimize the swelling of the porous structure when loaded with the cell suspension. However, it should be appreciated that the cross-linking for the second step is optional, insofar as the selection of the material for the MN scaffold provides sufficient strength and minimizes the swelling of the porous structure.

The loading of cells in this porous MN scaffold is achieved through dipping the tips of the MN scaffold in the cell-containing cryogenic medium. To maximize the viability of cells in this system, the formulation of the cryogenic medium is explored by fine-tubing the concentrations of DMSO and the hydrogel polymers (i.e. PEG, HES, MC, and CMC) (as shown by FIG. 4) and hMSCs is encapsulated for the wound healing assay (as shown by FIG. 6).

hMSCs is capable of allogeneic transplantation and well known for its excellent immunomodulation and tissue regeneration potentials.^{[16]} One attractive application is wound healing, where plenty of preclinical proofs validates MSCs biological functions in encouraging local fibroblasts migration and promoting vascularization^{[17]}. The hMSCs-loaded cryoMNs is developed and validated its benefits on wound healing using scratch assay and endothelial tube formation assay (FIG. 6). Due to the limitation of in vitro study, the above results mainly focus on the paracrine function of hMSCs. Other important therapeutic outcomes can only be examined on the animal model, including migration of released hMSCs, anti-inflammatory and immunomodulatory properties^{[18]}, remodeling of the extracellular matrix^{[19]}, and promoting skin appendages^{[20]}. The cryoMNs presented in this application largely protect the cell avoiding cell participation in the MN fabrication process while it shows the potential of cell transplantation for wound regeneration. Besides the cells, the simple and clean process (i.e. dip-loading and freezing) makes the porous MN scaffold a universal vehicle to shape the aqueous liquid formula in MN shape for non-invasive administration. Especially, this platform holds great potential for delivering, for example, thermal-sensitive biological agents (e.g., mRNA vaccines^{[21]}) that require ultralow temperature storage, which also alleviates concerns about activity loss during the fabrication process. It is believed that the current porous MN scaffold is achieved through lyophilization of crosslinked MeHA hydrogel inside PDMS mold, which has insufficient mechanical strength to achieve larger size of pore structures. For stronger materials such as ceramics or metals, it would be possible to generate higher porosity inside the structure for larger loading capacity.

In summary, embodiments of the present invention present a porous MN scaffold-assisted fast loading methods to minimize the steps from cell harvesting to cell delivery. The porous MN scaffold can rapidly absorb a therapeutic cell suspension and later frozen into solid status as cryoMNs. The cryoprotective medium forming the therapeutic cell suspension is formulated with a low concentration of DMSO and biocompatible polymers to achieve low toxicity while maintaining good cryoprotection for various types of cells (e.g., fibroblasts, HaCaT, A375, hMSCs, and melanocytes). Finally, it is found that the hMSC-loaded cryoMNs is fabricated as a proof-of-concept and demonstrated for its regenerative potential of encouraging fibroblasts migration and promoting angiogenesis on in vitro wound healing model.

Materials and Methods

300 kDa Sodium hyaluronic acid (300-HA, Mw 300 kDa) and 48 kDa sodium hyaluronic acid (48-HA, Mw 48 kDa) are purchased from Freda Biochem Co., Ltd. (Shandong, China, http://www.bloomagebioactive.com/). N, N-dimethylformamide (DMF, 227056), methacrylic anhydride (MAA, 276685), dimethyl sulfoxide (DMSO, 276855), sodium carboxymethyl cellulose (CMC, Mw 90 kDa, 419273), methyl cellulose (MC, viscosity 15 cP, M7140), hydroxyethyl starch (HES, medium Mw, Y0001277), poly(ethylene glycol) (PEG, Mw 10 kDa, P6667), chitosan (low Mw, 448869), gelatin (from bovine skin, G9391), Irgacure 2959, and alginic acid sodium salt (from brown algae, A0682) are purchased from Sigma-Aldrich (Singapore, Singapore, www.sigmaaldrich.com/). AlamarBlue™ cell viability assay, LIVE/DEAD™ viability/cytotoxicity kit, and CellTracker™ Green CMFDA are purchased from ThermoFisher Scientific (Waltham, Mass., USA, www.thermofisher.com/). All other materials except specifically mentioned are acquired from Sigma-Aldrich (above).

Synthesis of methacrylate modified hyaluronic acid (MeHA): HA is methacrylated by following the published protocol^{[8]}. Briefly, 1.0 g of HA (Mw=300 kDa or 48 kDa) is dissolved in the 50 mL of deionized water at 4° C. until complete dissolution. 33 mL DMF is then added into the HA solution to achieve the water/DMF mixture (3:2, v/v). 1.22 g MAA is subsequently added drop wisely into the solution while the pH is maintained at 8-9. The reaction is left overnight with continuous stirring at 4° C. Later, 2.46 g NaCl and pure ethanol are added sequentially to precipitate the product (i.e. MeHA). The crude product is collected through centrifuge and re-dissolved in deionized water. The purification of MeHA is conducted by dialysis against deionized water for 7 days. The purified product is obtained by lyophilization and stored at 4° C. MeHA is characterized by ¹H NMR spectroscopy (Bruker Avance II 300 MHz NMR) for modification degree.

Fabrication of porous MeHA MNs: The stainless-steel MN mold (Micropoint Technologies Pte Ltd, Singapore, Singapore, https://micropoint-tech.com/) has a base diameter (e.g., width as the base is circular in cross-sectional shape) of 300 μm, the tip radius of 5 μm, and a height of 1200 μm. To fabricate the negative mold, polydimethylsiloxane (PDMS, 10 mm thick, Dow Corning 184 Sylgard, Midland, Mich., USA, https://www.dow.com/) is poured over the master template to replicate the structure. After degassing by vacuum oven, the PDMS is cured at 70° C. for 1 hr and carefully peeled off from the template.

To fabricate the porous structure, MeHA (Mw=48 kDA or 300 kDa) is mixed with the photoinitiator (Irgacure 2959) at the mass ratio of 100:1 and dissolved in deionized water. The mixture is casted in the PDMS mold and centrifuged at 4,000 rpm for 5 mins to fill up the voids. Later, the patch is crosslinked by UV exposure. MNs with the different cross-linking degrees is named as CL3, CL5, CL10, and CL15, defined by their exposure time to UV for 3, 5, 10, 15 mins respectively. Then these MNs is frozen at −40° C. and lyophilized. The resulted porous MNs is carefully peeled off from the PMDS mold and crosslinked with UV for 15 mins to strengthen the scaffold matrix. The morphology and porous structures of MeHA MNs is examined by a microlens-equipped digital camera and field-emission scanning electron microscope (FESEM, JEOL JSM-6700, Tokyo, Japan, https://www.jeol.com/). The porosity of MNs is analyzed using ImageJ software. For each type of porous MNs, three independent images with 500× magnification are analyzed. The scaffold area is distinguished by the software, the porosity of each porous MN is calculated according to the following equation.

$$Porosity \% = \frac{{A}_{total} - {A}_{Scaffold}}{{A}_{total}} \times 100 \%$$

Where the Atotal and Ascaffold are the areas of the whole image and the scaffold part.

Cell culture: Human dermal fibroblasts (CellResearch Corporation Pte Ltd., Singapore, Singapore, https://www.cellresearchcorp.com/), human hypertrophic scar fibroblast (HSF, CellResearch Corporation Pte Ltd., above) human immortalized keratinocytes (HaCaT, Lonza, Basel, Switzerland, https://www.lonza.com/), human mesenchymal stem cells (hMSC, Lonza, above), and human malignant melanoma cell line (A375, ATCC are cultured in high-glucose Dulbecco's Modified Eagle's Medium (DMEM, Gibco, ThermoFisher Scientific, above) supplemented with 10% fetal bovine serum (FBS), 100 units/mL penicillin, and 100 μg/mL streptomycin. Human umbilical vein endothelial cells (HUVEC, Lonza, above) are cultured in EGM™-2 endothelial cell growth medium (EGM-2, Lonza, above) supplemented with 5% FBS. The cells are grown under 5% CO₂ at 37° with the medium replaced every two or three days.

Optimization of cryoprotective medium: Cryoprotective medium is prepared by dissolving polymers (HA, gelatin, alginic acid, chitosan, MC, CMC, PEG, and HES) and DMSO at the designed concentrations (0, 0.25, 0.5, 1.0, 2.5 wt. % for polymers; 0, 1, 2 v/v % by the volume of DMSO) in Dulbecco's Phosphate-Buffered Saline (DPBS, Gibco, ThermoFisher Scientific, above). Human dermal fibroblasts are used for testing the cryoprotective effect of the freezing medium. After the fibroblasts reached over 90% confluency, the cells are trypsinized and suspended in the freezing medium at the concentration of 2×10\hat{ }5 cells/ml. The cells are cryopreserved by gradient freezing and finally stored at −80° C. ultra-low freezer for 72 hrs. Later, the cells are thawed in a 37° C. water bath and seeded on the 96 well-plate and 48 well-plate using a fresh complete medium. After culturing for 24 hrs, the cells are imaged by phase contrast microscope and viability is accessed using the AlamarBlue™ cell viability assay. The cells cryopreserved in 10 v/v % DMSO under the same experimental condition are adopted as the positive control. The relative cell viability in each cryoprotective medium is calculated by normalized with positive control.

Fabrication of cryoMNs through dipping method with the porous MNs: The porous MN scaffolds are sterilized by UV for 30 mins before the operation. Cells (e.g., hMSCs) are firstly suspended in cryoprotective solutions at the density of 1×10⁶ cells/ml. The porous MNs are then soaked in the cell suspension with tip facing downwards for 1 min. The cell-loaded cryoMNs are placed inside the cryopreservation box and frozen under −80° C. for 1 day. The prepared cryoMNs are finally stored in liquid nitrogen.

Wound healing scratch assay: The capability of cell migration towards the wounded area is assessed using the scratch assay. The dermal fibroblasts are seeded in the bottom layer of the 48-well transwell (Costar®) at 90% confluency and cultured overnight. The linear defect on fibroblasts monolayer is created by scratching with a 1000 μL pipette tip. After scratching, the damaged monolayer is washed twice with the DPBS buffer and changed to 1.5 mL of low serum culture medium each well (i.e. 1% FBS for negative control group, and 5% FBS for positive control group, blank-cryoMN treatment, and hMSCs-loaded cryoMNs treatments group, n=3). For cryoMN treatment groups, each blank or hMSCs-loaded cryoMNs patches are dissolved in 200 μL culture medium and transferred into one transwell insert. For the positive control group, the low serum culture medium is supplemented with 20 ng/mL transforming growth factor (TGF)-β1 to accelerate fibroblasts proliferation. The bright-field image of the wounded area at the designed time points (i.e. 0, 24, 48 hr after treatment) is recorded with the Olympus IX71 inverted microscope. The width of the linear defect is measured using the ImageJ software. The wound closure is calculated by the following equation:

$$wound closure \% = ( 1 - \frac{wound width at 24 hr or 48 hr}{would width at 0 hr} ) \times 100 \%$$

Endothelial cell tube formation assay: The capability of hMSCs-loaded MNs in promoting angiogenesis is assessed using the endothelial cell tube formation assay. The HUVECs are per-stained with the CellTracker™ one night before the tube formation assay. The Matrigel® Matrix Basement Membrane (Corning Inc., Corning, N.Y., USA, https://www.corning.com/) is applied to coat the well plate. Briefly, 100 μL of Matrigel® Matrix is added to each well of the 48-well plate and solidified overnight at 37° C. Each well of tube formation contained 40,000 of the stained HUVECs in 300 μL of EGM-2 medium (1.5% FBS for negative control group, and 5% FBS for the positive control group, blank-cryoMN treatment, and hMSCs-loaded cryoMNs treatments group, n=3). In the positive control group, the EGM-2 medium is additionally supplemented with 40 ng/mL of VEGF to promote the formation of tubelike structures. For cryoMN treatment groups, one blank or hMSCs-loaded cryoMNs patches are dissolved in each well. After 24 hr incubation, the bright field and fluorescence images of each group are recorded with the Olympus IX71 inverted microscope. The total length of the tube, number of the tubes, and the branch points are measured using ImageJ to quantify the angiogenetic potential of each treatment.

Preparation and antigen stimulation of bone marrow-derived dendritic cells (BMDCs): BMDCs are isolated from bone marrow of C57BL/6 mice with previous reported protocol. Briefly, the femur bones are dissected, and the bone marrow is collected. Later the red blood cells are lysed using ACK lysing buffer (Gibco, ThermoFisher Scientific, above). The cells are resuspended at the concentration of 1×10\hat{ }6 cells/ml and cultured in completed RPMI 1640 medium supplemented with GM-CSF (PeproTech Inc., Cranbury, USA, https://www.peprotech.com/gb/) and IL-4 (PeproTech Inc., above) and replaced with fresh medium every 2 days. On the 7 day, non-adherent and loosely adherent cells are collected and pulsed with 100 μg/mL LPS and 50 μg/mL OVA for 24 hours to obtain LPS-pulsed and OVA-pulsed DCs, respectively.

In vivo vaccination using OVA-DCs loaded cryoMNs: The experiment is performed on C57BL/6 mice (male, 6-8 weeks) in accordance with ethical approval by the Animal Research Ethics Sub-Committee of City University of Hong Kong with reference no. of A-0493. The animals are purchased from the Laboratory Animal Research Unit of City University of Hong Kong (Hong Kong, S.A.R., http://www.cityu.edu.hk/laru/). The mice are housed in ventilated caging systems in a 12:12 h LD cycle at constant temperature and humidity. The mice are randomly allocated for three treatment groups: untreated, s.c. injection group and OVA-DCs loaded cryoMNs group. Each mouse receives vaccinations twice a week for a total eight doses, in which 2×10\hat{ }5 OVA-DCs are administrated in each treatment. At day 28, the mice are sacrificed and their lymph nodes, spleens, and major organs collected. The lymphocytes from excised lymph nodes are stained for surface markers (i.e., CD11c, MHCII, and CD86) of mature DCs to evaluate the homing effects of the delivered OVA-DCs vaccines. The splenocytes are obtained from excised spleens and restimulated using OVA to evaluate the specific proliferation with presence of model antigen. The proliferation assay is performed using AlamarBlue™ viability assay following the manufacturer's protocol.

Determination of OVA-specific immunoglobulin levels using ELISA assay: The tail vein blood was collected, and serum was separated by centrifugation using 1000 g for 15 minutes at 4° C. Briefly, Nunc™ MaxiSorb™ ELISA plate (BioLegend Inc., San Diego, USA, https://www.biolegend.com/) is coated with 50 μL 5 μg/mL OVA for 16-18 hours and later is blocked with 1.0% BSA for 1 hour at room temperature. 504 of diluted serum (1:100) is added into each well of the coated plate. After 1 hour incubation and wash, HRP Goat anti-mouse IgG (1:2000 dilution, BioLegend Inc., above) is added into the plate and incubated for 1 hour. The TMB substrate is added to develop color and later quantified by the absorbance at 450 nm (SpectraMAX® M5e Microplate Reader, Molecular Devices, San Jose, USA, https://www.moleculardevices.com/).

Statistical analysis: Each experiment is repeated at least three times in triplicate unless specified otherwise. Student's T-test is used to determine p-values, with p<0.05 considered significant. *p<0.05, **p<0.01, ***p<0.001, and ****p<0.0001.

### REFERENCES

(1) Chua, J. Y.; Pendharkar, A. V.; Wang, N.; Choi, R.; Andres, R. H.; Gaeta, X.; Zhang, J.; Moseley, M. E.; Guzman, R. Intra-Arterial Injection of Neural Stem Cells using a Microneedle Technique does not Cause Microembolic Strokes. Journal of Cerebral Blood Flow & Metabolism 2011, 31 (5), 1263-1271. DOI: 10.1038/jcbfm.2010.213.
(2) Gualeni, B.; Coulman, S. A.; Shah, D.; Eng, P. F.; Ashraf, H.; Vescovo, P.; Blayney, G. J.; Piveteau, L.-D.; Guy, O. J.; Birchall, J. C. Minimally invasive and targeted therapeutic cell delivery to the skin using microneedle devices. British Journal of Dermatology 2018, 178 (3), 731-739. DOI: https://doi.org/10.1111/bjd.15923.
(3) Lee, K.; Xue, Y.; Lee, J.; Kim, H.-J.; Liu, Y.; Tebon, P.; Sarikhani, E.; Sun, W.; Zhang, S.; Haghniaz, R.; et al. A Patch of Detachable Hybrid Microneedle Depot for Localized Delivery of Mesenchymal Stem Cells in Regeneration Therapy. Advanced Functional Materials 2020, 30 (23), 2000086. DOI: https://doi.org/10.1002/adfm.202000086.
(4) Chen, Y.-H.; Lin, D.-C.; Chern, E.; Huang, Y.-Y. The use of micro-needle arrays to deliver cells for cellular therapies. Biomedical Microdevices 2020, 22 (4), 63. DOI: 10.1007/s10544-020-00518-z.
(5) Gao, Y.; Hou, M.; Yang, R.; Zhang, L.; Xu, Z.; Kang, Y.; Xue, P. Highly Porous Silk Fibroin Scaffold Packed in PEGDA/Sucrose Microneedles for Controllable Transdermal Drug Delivery. Biomacromolecules 2019, 20 (3), 1334-1345. DOI: 10.1021/acs.biomac.8b01715.
(6) Liu, L.; Kai, H.; Nagamine, K.; Ogawa, Y.; Nishizawa, M. Porous polymer microneedles with interconnecting microchannels for rapid fluid transport. RSC Advances 2016, 6 (54), 48630-48635, 10.1039/C6RA07882F. DOI: 10.1039/C6RA07882F. Li, J.; Liu, B.; Zhou, Y.; Chen, Z.; Jiang, L.; Yuan, W.; Liang, L. Fabrication of a Ti porous microneedle array by metal injection molding for transdermal drug delivery. PLoS ONE 2017, 12, e0172043, Report. (accessed 2020/5/9/). From Gale Gale OneFile: Health and Medicine. van der Maaden, K.; Lunge, R.; Vos, P. J.; Bouwstra, J.; Kersten, G.; Ploemen, I. Microneedle-based drug and vaccine delivery via nanoporous microneedle arrays. Drug Delivery and Translational Research 2015, 5 (4), 397-406. DOI: 10.1007/s13346-015-0238-y.
(7) Tekko, I. A.; Permana, A. D.; Vora, L.; Hatahet, T.; McCarthy, H. O.; Donnelly, R. F. Localised and sustained intradermal delivery of methotrexate using nanocrystal-loaded microneedle arrays: Potential for enhanced treatment of psoriasis. European Journal of Pharmaceutical Sciences 2020, 152, 105469. DOI: https://doi.org/10.1016/j.ejps.2020.105469. Bok, M.; Lee, Y.; Park, D.; Shin, S.; Zhao, Z.-J.; Hwang, B.; Hwang, S. H.; Jeon, S. H.; Jung, J.-Y.; Park, S. H.; et al. Microneedles integrated with a triboelectric nanogenerator: an electrically active drug delivery system. Nanoscale 2018, 10 (28), 13502-13510, 10.1039/C8NR02192A. DOI: 10.1039/C8NR02192A.
(8) Hao, C.; Mengjia, Z.; Xiaojun, Y.; Aung, T.; Z., S. R.; Rongjie, K.; Jingqi, T.; Phan, K. D.; Linbo, L.; Peng, C.; et al. A Swellable Microneedle Patch to Rapidly Extract Skin Interstitial Fluid for Timely Metabolic Analysis. Advanced Materials 2017, 29 (37), 1702243. DOI: doi:10.1002/adma.201702243.
(9) Hou, Q.; Grijpma, D. W.; Feijen, J. Porous polymeric structures for tissue engineering prepared by a coagulation, compression moulding and salt leaching technique. Biomaterials 2003, 24 (11), 1937-1947. DOI: https://doi.org/10.1016/S0142-9612(02)00562-8.
(10) Bai, Y.-X.; Li, Y.-F. Preparation and characterization of crosslinked porous cellulose beads. Carbohydrate Polymers 2006, 64 (3), 402-407. DOI: https://doi.org/10.1016/j.carbpol.2005.12.009.
(11) Hey, J.; MacFarlane, D. Crystallization of ice in aqueous solutions of glycerol and dimethyl sulfoxide. 1. A comparison of mechanisms. Cryobiology 1996, 33 (2), 205-216. Gao, D.; Critser, J. Mechanisms of cryoinjury in living cells. ILAR journal 2000, 41 (4), 187-196.
(12) Deller, R. C.; Vatish, M.; Mitchell, D. A.; Gibson, M. I. Synthetic polymers enable non-vitreous cellular cryopreservation by reducing ice crystal growth during thawing. Nature Communications 2014, 5, 3244, Article. Deller, R. C.; Vatish, M.; Mitchell, D. A.; Gibson, M. I. Glycerol-Free Cryopreservation of Red Blood Cells Enabled by Ice-Recrystallization-Inhibiting Polymers. ACS Biomaterials Science & Engineering 2015, 1 (9), 789-794. DOI: 10.1021/acsbiomaterials.5b00162.
(13) Naaldijk, Y.; Staude, M.; Fedorova, V.; Stolzing, A. Effect of different freezing rates during cryopreservation of rat mesenchymal stem cells using combinations of hydroxyethyl starch and dimethylsulfoxide. BMC Biotechnology 2012, 12 (1), 49. DOI: 10.1186/1472-6750-12-49. Imaizumi, K.; Nishishita, N.; Muramatsu, M.; Yamamoto, T.; Takenaka, C.; Kawamata, S.; Kobayashi, K.; Nishikawa, S.-i.; Akuta, T. A Simple and Highly Effective Method for Slow-Freezing Human Pluripotent Stem Cells Using Dimethyl Sulfoxide, Hydroxyethyl Starch and Ethylene Glycol. PLOS ONE 2014, 9 (2), e88696. DOI: 10.1371/journal.pone.0088696. Wang, H.-Y.; Lun, Z.-R.; Lu, S.-S. Cryopreservation of umbilical cord blood-derived mesenchymal stem cells without dimethyl sulfoxide. CryoLetters 2011, 32 (1), 81-88.
(14) Ashwood-Smith, M.; Warby, C.; Connor, K.; Becker, G. Low-temperature preservation of mammalian cells in tissue culture with polyvinylpyrrolidone (PVP), dextrans, and hydroxyethyl starch (HES). Cryobiology 1972, 9 (5), 441-449. Stolzing, A.; Naaldijk, Y.; Fedorova, V.; Sethe, S. Hydroxyethylstarch in cryopreservation—Mechanisms, benefits and problems. Transfusion and Apheresis Science 2012, 46 (2), 137-147. DOI: https://doi.org/10.1016/j.transci.2012.01.007. Asada, M.; Ishibashi, S.; Ikumi, S.; Fukui, Y. Effect of polyvinyl alcohol (PVA) concentration during vitrification of in vitro matured bovine oocytes. Theriogenology 2002, 58 (6), 1199-1208.
(15) Chang, H.; Chew, S. W. T.; Zheng, M.; Lio, D. C. S.; Wiraja, C.; Mei, Y.; Ning, X.; Cui, M.; Than, A.; Shi, P.; et al. Cryomicroneedles for transdermal cell delivery. Nature Biomedical Engineering 2021, 5 (9), 1008-1018. DOI: 10.1038/s41551-021-00720-1.
(16) Parekkadan, B.; Milwid, J. M. Mesenchymal stem cells as therapeutics. Annual review of biomedical engineering 2010, 12, 87-117.
(17) Yaojiong, W.; Liwen, C.; G., S. P.; E., T. E. Mesenchymal Stem Cells Enhance Wound Healing Through Differentiation and Angiogenesis. STEM CELLS 2007, 25 (10), 2648-2659. DOI: doi:10.1634/stemcells.2007-0226.
(18) Formigli, L.; Paternostro, F.; Tani, A.; Mirabella, C.; Quattrini Li, A.; Nosi, D.; D'Asta, F.; Saccardi, R.; Mazzanti, B.; Lo Russo, G.; et al. MSCs seeded on bioengineered scaffolds improve skin wound healing in rats. Wound Repair and Regeneration 2015, 23 (1), 115-123. DOI: https://doi.org/10.1111/wrr.12251. Maxson, S.; Lopez, E. A.; Yoo, D.; Danilkovitch-Miagkova, A.; LeRoux, M. A. Concise Review: Role of Mesenchymal Stem Cells in Wound Repair. STEM CELLS Translational Medicine 2012, 1 (2), 142-149. DOI: https://doi.org/10.5966/sctm.2011-0018.
(19) Fu, X.; Fang, L.; Li, X.; Cheng, B.; Sheng, Z. Enhanced wound-healing quality with bone marrow mesenchymal stem cells autografting after skin injury. Wound Repair and Regeneration 2006, 14 (3), 325-335. Jeon, Y. K.; Jang, Y. H.; Yoo, D. R.; Kim, S. N.; Lee, S. K.; Nam, M. J. Mesenchymal stem cells' interaction with skin: Wound-healing effect on fibroblast cells and skin tissue. Wound repair and regeneration 2010, 18 (6), 655-661.
(20) Li, H.; Fu, X.; Ouyang, Y.; Cai, C.; Wang, J.; Sun, T. Adult bone-marrow-derived mesenchymal stem cells contribute to wound healing of skin appendages. Cell and tissue research 2006, 326 (3), 725-736.
(21) Holm, M. R.; Poland, G. A. Critical aspects of packaging, storage, preparation, and administration of mRNA and adenovirus-vectored COVID-19 vaccines for optimal efficacy. Vaccine 2021, 39 (3), 457-459. DOI: 10.1016/j.vaccine.2020.12.017 PubMed.

Unless otherwise defined, all terms (including technical and scientific terms) used herein have the same meaning as commonly understood by one of ordinary skill in the art to which the embodiments of the invention belong. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

While the disclosure has been described with reference to an exemplary embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

All references specifically cited herein are hereby incorporated by reference in their entireties. However, the citation or incorporation of such a reference is not necessarily an admission as to its appropriateness, citability, and/or availability as prior art to/against the present invention.

1. A method for fabricating a cryomicroneedle, comprising:
(A) providing a microneedle scaffold comprising a plurality of pores;
(B) providing a suspension comprising a biological agent;
(C) loading the biological agent into the microneedle scaffold by immersing the microneedle scaffold in the suspension to form a loaded microneedle scaffold; and
(D) freezing the loaded microneedle scaffold to provide the cryomicroneedle.

2. The method according to claim 1, wherein the microneedle scaffold is made by a process comprising:
(A) providing a mold comprising a plurality of voids;
(B) providing a scaffold precursor solution;
(C) casting the scaffold precursor solution into the mold;
(D) filling the plurality of voids with the scaffold precursor;
(E) cross-linking the scaffold precursor solution to form a scaffold comprising a plurality of pores;
(F) lyophilizing the scaffold;
(G) demoulding the scaffold from the mold to form a microneedle scaffold; and
(H) optionally, cross-linking the microneedle scaffold.

3. The method according to claim 1, wherein the microneedle scaffold comprises a hydrogel, an aerogel, a biodegradable polymer, a metal, a bioceramic and a combination thereof.

4. The method according to claim 2, wherein the scaffold precursor solution comprises a precursor selected from the group consisting of hyaluronic acid, agarose, alginic acid, chitosan, cellulose, dextran, fibrin, gelatin/collagen, poly(ethylene glycol) (PEG), poly(ethylene oxide) (PEO), polyacrylamide (PAA), poly(vinyl alcohol) (PVA), polyglycolic acid (PGA), polylactic acid (PLA), stainless steel, titanium, aluminum, alumina, clay, silicon dioxide, zirconia, calcium carbonate, calcium sulfate-based ceramic, a calcium phosphate-based ceramic, a derivative thereof, an alloy thereof, and a combination thereof.

5. The method according to claim 1, wherein the cross-linking of the scaffold precursor solution comprises the step of exposing the scaffold precursor solution to a condition selected from the group consisting of light exposure, radiation exposure, copolymerization initiation, a thaw-freeze cycle, reduced temperature, ionic solution exposure, pH adjustment, thermal curing, solvent-induced phase inversion, sintering, and a combination thereof.

6. The method according to claim 1, wherein the cross-linking of the microneedle scaffold comprises the step of exposing the microneedle scaffold to a condition selected from the group consisting of light exposure, radiation exposure, copolymerization initiation, a thaw-freeze cycle, reduced temperature, ionic solution exposure, pH adjustment, thermal curing, solvent-induced phase inversion, sintering, and a combination thereof.

7. The method according to claim 1, wherein the microneedle scaffold comprises a microneedle scaffold material selected from the group consisting of a protein, a nucleic acid, a ceramic, a metal and a combination thereof.

8. The method according to claim 1, wherein the microneedle scaffold has a needle length of from about 25 μm to about 2000 μm.

9. The method according to claim 1, wherein the microneedle scaffold has a needle base width of from about 10 μm to about 750 μm.

10. The method according to claim 1, wherein the suspension further comprises a cryoprotective agent selected from the group consisting of a cell membrane-penetrating cryoprotectant, a non-penetrating cryoprotectant, and a combination thereof.

11. The method according to claim 10, wherein:
(A) the cell membrane-penetrating cryoprotectant is selected from the group consisting of dimethyl sulfoxide (DMSO), methanol, butanediol, proline glycerol, ethylene glycol, propylene glycol, diethylene glycol, triethylene glycol, glyceryl glucoside, formamide, acetamide, dimethylacetamide, trimethylamine, a cell-penetrating zwitterionic cryoprotectant and a combination thereof; and
(B) the non-penetrating cryoprotectant is selected from the group consisting of a non-permeable zwitterionic cryoprotectant, a polymeric cryoprotectant and a combination thereof; or a polymeric cryoprotectant selected from the group consisting of polyethylene glycol (PEG), polyvinyl pyrrolidone, polyvinyl alcohol (PVA), hydroxyethyl starch (HES), methyl cellulose (MC), carboxymethyl cellulose (CMC), dextran, polyproline, hyaluronic acid, alginic acid, carboxylated poly-L-lysine, poly(ampholytes) and a combination thereof.

12. The method according to claim 11, wherein the zwitterionic cryoprotectant comprises betaine.

13. The method according to claim 1, wherein the biological agent is selected from the group consisting of a cell organoid, a cell aggregate, a cell, a bacteria, a virus, a protein/peptide, a nucleic acid/DNA/RNA, a cell extract or component, a cell-mimicking particle, a vector, and a combination thereof.

14. A cryomicroneedle prepared according to the method according to claim 1.

15. A cryomicroneedle according to claim 14, wherein the biological agent comprises a human cell.

16. A method for autologous cell therapy comprising the steps of:
(A) providing a subject for the autologous cell therapy;
(B) collecting a biological sample from the subject, wherein the biological sample comprises a cell from the subject;
(C) engineering the biological sample into the biological agent that comprising and/or not comprising a cell;
(D) multiplying the biological agent ex vivo;
(E) providing a cryomicroneedle according to claim 14 comprising the biological agent; and
(F) returning the biological agent to the subject.

17. The method according to claim 16, wherein the biological agent comprises a human cell.

18. A vaccination method for vaccinating a subject comprising the steps of:
(A) providing a subject;
(B) providing a cryomicroneedle according to claim 14 comprising the biological agent, wherein the biological agent comprises a vaccine; and
(C) injecting the subject with the biological agent via the cryomicroneedle.

19. The vaccination method according to claim 18, wherein the subject is a mammal.

20. The vaccination method according to claim 19, wherein the subject is a human.
